(19) United States
(12) Patent Application Publication
(10) Pub. No.: US 2007/0054348 A1
(43) Pub. Date: Mar. 8, 2007

(54) Methods of screening bifunctional molecules for modulated pharmacokinetic properties

(76) Inventors: Jason E. Gestwicki, Ann Arbor, MI (US); Gerald R. Crabtree, Woodside, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/512,721

(22) Filed: Aug. 29, 2006

Related U.S. Application Data
(60) Provisional application No. 60/713,211, filed on Aug. 30, 2005.

(51) Int. Cl. C12Q 1/26

(52) U.S. Cl. 435/25

(57) ABSTRACT

Methods for screening bifunctional molecules of a drug of interest for modulated pharmacokinetic properties are provided. The subject methods include combining in a reaction mixture a metabolizer of the drug of interest, a reporter of activity of the metabolizer; and a bifunctional compound of the drug of interest. Signal from the reporter is then-evaluated to determine whether the bifunctional compound has a modulated pharmacokinetic property as compared to a free drug control. Also provided are kits and devices for practicing the subject methods of the invention.

### CROSS-REFERENCE

This application claims the benefit of U.S. Provisional Application No. 60/713,211 filed Aug. 30, 2005, which application is incorporated herein by reference.

### GOVERNMENT RIGHTS

This invention was made with government support under federal grant nos. NS046789 awarded by the National Institutes of Health. The United States Government may have certain rights in this invention.

### BACKGROUND OF THE INVENTION

Any chemical agent that affects any process of living is a drug. Drugs are a critical tool for health care practitioners, as they are used in the prevention, diagnosis and treatment of disease. Because of their criticality to the health care profession, annual world investment into the research and development of new chemical agents with therapeutic potential reaches into the billions of dollars. As a result, a large number of drugs have been developed to date and new chemical agents having potential therapeutic utility are frequently discovered. Chemical agents that find, or have found, use as drugs include naturally occurring and synthetic small molecules, as well as larger molecules, such as proteinaceous compounds.

A major challenge in the development of drugs is the predictable modulation of pharmacokinetic properties. Major pharmacokinetic parameters that effect the ability of a particular drug to treat a given condition include: the drug half-life, the hepatic first-pass metabolism of the drug, the volume of distribution of the drug, the degree of albumin binding of the drug, etc. Each of the above parameters can have a profound effect on the efficacy of a given drug agent.

As such, of great interest to the pharmaceutical industry and related fields would be the development of methods for screening compounds for at least one modulated pharmacokinetic property. The present invention addresses this need.

Relevant Literature

Patent publications of interest include: WO 91/01743; WO 94/18317; WO 95/02684; WO 95/10302; WO 96/06111; WO 96/12796; WO 96/13613; WO 97/25074; WO 97/29372; WO 98/11437; WO 98/47916; U.S. Pat. No. 5,714,142; U.S. Pat. No. 5,830,462; U.S. Pat. No. 5,843,440; U.S. Pat. No. 5,871,753; U.S. Pat. No. 6,887,842; U.S. Pat. No. 6,372,712; and U.S. Pat. No. 6,921,531.

References of interest include: Briesewitz et al., Proc. Nat'l Acad. Sci. USA (March 1999) 96: 1953-1958; Clardy, Proc. Nat'l Acad. Sci. USA (March 1999) 1826-1827; Crabtree & Schreiber, Elsevier Trends Journal (November 1996) 418-422; Spencer et al., Curr. Biol. (July 1996) 6:839-847; Spencer et al., Science (1993) 262: 1019; Chakraborty et al., Chem. & Biol. (March 1995) 2:157-161; Ho et al., Nature (1996) 382: 822; Riviera et al., Nature Medicine (1996) 2: 1028; Klemm et al., Current Biology (1997) 7: 638; Belshaw et al., Proc. Nat'l. Acad. Sci. USA (1996) 93: 4604; Livnah et al., Science (1996) 273: 464; Johnson et al., Chemistry and Biology, (1997) 4: 939; Garboczi et al., Nature (1996) 384:134; Kissenger et al., Nature (1995) 378:641; Griffith et al., Cell (1995) 82: 507; Choi et al., Science (1996) 273:239; Braun et al., J. Am. Chem. Soc. (2003) 125:7575; Gestwicki et al., Science (2004) 306:865. Also of interest are Kramer et al., J. Biol. Chem. (1992) 267:18598-18604; and Varshavsky, Proc. Nat'l Acad. Sci. USA (March 1998) 95: 2094-2099; Varshavsky, Proc. Nat'l Acad. Sci. USA (April 1995) 92:3663-3667; and Mu et al., Biochem. Biophys. Res. Comm. (1999)255:75-79; Kumar et al., Bioconj. Chem. (2001) 12:464; Zhuang et al., J. Med. Chem. (2001) 44:1905; Zim et al., Org. Lett. (2003) 5:2413; and Wang et al., J. Mol. Neurosci. (2002) 19:11.

### SUMMARY OF THE INVENTION

Methods for screening bifunctional molecules of a drug for modulated pharmacokinetic properties are provided. The subject methods include combining in a reaction mixture a metabolizer of the drug of interest, a reporter for the activity of the metabolizer; and a bifunctional compound that includes the drug of interest. Signal from the reporter is then evaluated to determine whether the bifunctional compound of the drug has a modulated pharmacokinetic property, e.g., as compared to a free drug control. Also provided are kits and devices for practicing the subject methods of the invention.

These and other objects, advantages, and features of the invention will become apparent to those persons skilled in the art upon reading the details of the invention as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1A is a schematic representation of an exemplary bifunctional molecule that includes a drug moiety that is capable of binding to a target of interest joined to FK506 by a linker.

FIG. 1B is a schematic diagram of a proposed scheme of protection of a drug compound by associating the compound with a pharmacokinetic modulating moiety, such as FK506.

FIG. 1C is a schematic diagram of controlled distribution of a drug moiety of a bifunctional compound as compared to the same unprotected drug.

FIG. 2A shows the results of a cytochrome p450 3A4 (CYP3A4) assay for the bifunctional compound Curcumin-FK-506 in the presence and absence of rhFKBP12 (left panel) and a plot of the initial rate of degradation and the change in Km and Vmax (right panel).

FIG. 2B shows the results of a cytochrome p450 3A4 (CYP3A4) assay for the bifunctional compound TZDM-SLF.

FIG. 3 is a set of images showing the biodistribution of the bifunctional compound TZDM-SLF in COS-1 cells by fluorescence microscopy as compared to the free TZTM compound.

FIG. 4 shows the results of a cytochrome p450 3A4 (CYP3A4) assay for the bifunctional compound Curcumin-FK-506 in Chinese hamster ovary cells.

FIG. 5 shows the results of a cytochrome p450 3A4 (CYP3A4) assay for the naturally bifunctional compound FK-506 in the presence and absence of rhFKBP12.

### DEFINITIONS

The term “bifunctional molecule” refers to a non-naturally occurring molecule that includes a pharmacokinetic modulating moiety and a drug moiety, where these two components may be covalently bonded to each other either directly or through a linking group.

The term “drug” refers to any active agent that affects any biological process. Active agents which are considered drugs for purposes of this application are agents that exhibit a pharmacological activity. Examples of drugs include active agents that are used in the prevention, diagnosis, alleviation, treatment or cure of a disease condition.

By “pharmacologic activity” is meant an activity that modulates or alters a biological process so as to result in a phenotypic change, e.g. cell death, cell proliferation etc.

By “pharmacokinetic property” is meant a parameter that describes the disposition of an active agent in an organism or host. Representative pharmacokinetic properties include: drug half-life, hepatic first-pass metabolism, volume of distribution, degree of blood serum protein, e.g. albumin, binding, etc.

By “half-life” is meant the time for one-half of an administered drug to be eliminated through biological processes, e.g. metabolism, excretion, etc.

By “hepatic first-pass metabolism” is meant the propensity of a drug to be metabolized upon first contact with the liver, i.e. during its first pass through the liver.

By “volume of distribution” is meant the distribution and degree of retention of a drug throughout the various compartments of an organism, e.g. intracellular and extracellular spaces, tissues and organs, etc.

By “degree of blood serum binding” is meant the propensity of a drug to be bound by a blood serum protein, such as albumin, in manner such that the activity of the drug is substantially dissipated if not abolished. This property is also referred to herein as the blood serum binding effect. In those embodiments where the blood serum protein is albumin, this property is also referred to as the albumin binding effect.

The term “efficacy” refers to the effectiveness of a particular active agent for its intended purpose, i.e. the ability of a given active agent to cause its desired pharmacologic effect.

### DETAILED DESCRIPTION OF THE INVENTION

Methods for screening bifunctional molecules of a drug for modulated pharmacokinetic properties are provided. The subject methods include combining in a reaction mixture a metabolizer of the drug of interest, a reporter for the activity of the metabolizer; and a bifunctional compound of the drug of interest. Signal from the reporter is then evaluated to determine whether the bifunctional compound of the drug has a modulated pharmacokinetic property, e.g., as compared to a free drug control. Also provided are kits and devices for practicing the subject methods of the invention.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods

As summarized above, aspects of the present invention include methods of screening a bifunctional molecule of a drug (described in greater detail below) to determine whether the bifunctional molecule of the drug includes some desirable modulated pharmokokinetic profile, e.g., as compared to a reference, such as a free drug control. In practicing the subject methods, candidate bifunctional molecules can be screened for those molecules that exhibit the desired modulated pharmacokinetic profile, e.g. desired half-life, desired hepatic first-pass metabolism, desired volume of distribution and/or desired degree of albumin binding. In representative embodiments, the subject screening methods are used to determine whether a candidate bifunctional molecule that includes a drug moiety exhibits at least one modulated pharmacokinetic property as compared to a free drug control. By “free drug control” is meant a drug compound or an active derivative thereof that is not associated with a pharmacokinetic modulating moiety as a bifunctional molecule.

In general, any convenient screening assay may be employed, where the particular screening assay may be one known to those of skill in the art or one developed in view of the specific molecule and property being studied. Representative embodiments of the subject methods include first producing a reaction mixture of at least: a metabolizer of the drug of interest; a reporter of the activity the metabolizer, and a candidate bifunctional molecule of the drug interest. Once the reaction mixture is produced, signal from the reporter is evaluated to determine whether the bifunctional compound has a modulated pharmacokinetic property, e.g., as compared to a free drug control.

As used herein, the phrase “metabolizer of the drug of interest” refers to an entity, e.g., molecule or complex of molecules, that participates in the chemical breakdown or buildup of a drug of interest in a manner that modulates the activity of the drug of interest. In representative embodiments, a metabolizer is a compound that modulates the pharmacokinetic properties of a drug in a host, such as half-life of the drug or hepatic-first pass metabolism of the drug. In representative embodiments, the metabolizer modulates the pharmacokinetic properties of a drug in a host by, for example, decreasing the half-life of the drug, e.g., by increasing the hepatic-first pass metabolism of the drug. A representative metabolizer of interest is a member of the cytochrome p450 mixed-function oxidase system involved in the metabolism of xenobiotics (e.g., drug compounds) in the body (i.e., a CYP compound). Examples of CYP compounds of interest include, but are not limited to, the commercially available CYP1A2, CYP2B6, CYP2C9, CYP2C19, CYP2D6, CYP2E1, CYP3A4, and CYP3A5.

As used herein a “reporter” refers to compound that interacts with the metabolizer in a manner sufficient to provide a signal that is indicative or representative of the activity of the metabolizer. For example, where the metabolizer is an enzyme, the reporter may be a substrate of the metabolizer enzyme. In general, the reporter produces a detectable signal upon interaction, e.g., binding, with the metabolizer. In representative embodiments, the signal generated by the reporter is proportional to the activity of the metabolizer, and may be directly or inversely proportional to the metabolizer activity. The signal provided by the reporter may be any of a number of different types of signals, including but not limited to: optical signals, including visual signals, chemical signals and the like.

In representative embodiments, the reporter is a compound that generates an optical signal, e.g., in the visual or non-visual spectrum, which is proportional to the activity of the metabolizer. In certain of the these embodiments, the reporter is a substrate for an enzyme metabolizer, such as a CYP as reviewed above, that generates a fluorescent signal that is proportional to the activity the substrate. In certain of these embodiments, the reporter is a fluorogenic substrate of the metabolizer that generates a fluorescent signal that is directly proportional to the activity of the metabolizer on the substrate. For example, the reporter of certain of these embodiments is a fluorescent substrate of the metabolizer that is in a blocked state such that it is non-fluorescent until it is acted on by the metabolizer to remove the blocking moiety and thereby provide for a fluorescent signal. In these representative embodiments, the blocked fluorescent substrate is metabolized by the metabolizer resulting in an unblocked fluorescent substrate that is fluorescent. Over time, as more blocked fluorescent substrate is metabolized the fluorescence of the reaction increases, thereby providing for a signal that is directly proportional to the activity of the metabolizer.

In some embodiments, the reporter substrate of the compound is a fluorogenic CYP substrate. Suitable fluorogenic CYP substrates include the commercial available VIVID® fluorogenic CYP substrates. The fluorogenic CYP substrate will be selected based on the CYP compound selected for use in the assay. Exemplary fluorogenic CYP substrates and their corresponding CYP compound are provided in Table 1.

TABLE 1

| CYP | Fluorogenic CYP Substrate |
|---|---|
| CYP1A2 | VIVID ® EOMCC (Blue) |
| CYP2B6 | VIVID ® BOMCC (Blue) |
| | VIVID ® BOMFC (Cyan) |
| CYP2C9 | VIVID ® BOMCC (Blue) |
| | VIVID ® BOMF (Green) |
| | VIVID ® OOMR (Red) |
| CYP2C19 | VIVID ® EOMCC (Blue) |
| CYP2D6 | VIVID ® EOMCC (Blue) |
| | VIVID ® MOBFC(Cyan) |
| CYP2E1 | VIVID ® EOMCC (Blue) |
| CYP3A4 | VIVID ® BOMCC (Blue) |
| | VIVID ® BOMFC (Cyan) |
| | VIVID ® DBOMF (Green) |
| | VIVID ® BOMR (Red) |
| CYP3A5 | VIVID ® BOMCC (Blue) |
| | VIVID ® BOMFC (Cyan) |
| | VIVID ® DBOMF (Green) |

In certain embodiments, the reporter and metabolizer for a given assay are selected such that the drug of interest and the reporter compete to be metabolized by the metabolizer, e.g., they are both substrates for the metabolizer such that if the metabolizer is metabolizes drug it is not metabolizing reporter at the same time, and vice versa. As such, a competitive format is employed such that both the drug (or candidate bifunctional molecule thereof) and the reporter compete for being metabolized by the metabolizer.

Also present in the reaction mixture of many embodiments of the subject methods is a pharmacokinetic modulating protein (PMP). The specific PMP that is present in these embodiments will be dependent on the pharmacokinetic modulating moiety of the candidate bifunctional molecule, described in greater detail below. In some embodiments, where one wishes to modulate the half-life, hepatic first-pass metabolism, or volume of distribution, intracellular proteins are often of interest, where representative intracellular proteins of interest include, but are not limited to: peptidyl-prolyl isomerases, e.g. FKBPs and cyclophilins; ubiquitously expressed molecular chaperones, e.g. Heat Shock Protein 90 (Hsp90); steroid hormone receptors, e.g. estrogen receptors, glucocorticoid receptors, androgen receptors; retinoic acid binding protein, cytoskeletal proteins, such as tubulin and actin; etc. In other embodiments, where one wishes to modulate the half-life, hepatic first-pass metabolism, or volume of distribution, extracellular proteins are also of interest, where representative extracellular proteins of interest include, but are not limited to: enzymes,. e.g. kinases, phosphatases, reductases, cyclooxygenases, proteases and the like, targets comprising domains involved in protein-protein interactions, such as the SH2, SH3, PTB and PDZ domains, structural proteins, e.g. actin, tubulin, etc., membrane receptors, immunoglobulins, e.g. IgE, cell adhesion receptors, such as integrins, etc, ion channels, transmembrane pumps, transcription factors, signaling proteins, and the like.

Of particular interest as intracellular pharmacokinetic modulating proteins are cis-trans peptidyl-prolyl isomerases which interact with many proteins because of their chaperonin/isomerase activity, e.g. FKBPs and cyclophilins. Peptidyl-prolyl isomerases of interest include FKBPs. A number of different FKBPs are known in the art, and include those described in: Sabatini et al., Mol. Neurobiol. (October 1997) 15:223-239; Marks, Physiol. Rev. (July 1996) 76:631-649; Kay, Biochem J. (March, 1996) 314: 361-385; Braun et al., FASEB J. (January 1995) 9:63-72; Fruman et al, FASEB J. (April 1994) 8:391-400; and Hacker et al., Mol. Microbiol. (November 1993) 10: 445-456. FKBPs of interest include FKBP 12, FKBP 52, FKBP 14.6 (described in U.S. Pat. No. 5,525,523, the disclosure of which is herein incorporated by reference); FKBP 12.6 (described in U.S. Pat. No. 5,457,182 the disclosure of which is herein incorporated by reference); FKBP 13 (described in U.S. Pat. No. 5,498,597, the disclosure of which is herein incorporated by reference); and HCB (described in U.S. Pat. No. 5,196,352 the disclosure of which is herein incorporated by reference); where FKBP 12 and FKBP 52 are of particular interest as intracellular pharmacokinetic modulating proteins.

Also of specific interest as intracellular PMPs are cyclophilins. A number of cyclophilins are known in the art and are described in Trandinh et al., FASEB J. (December 1992) 6: 3410-3420; Harding et al., Transplantation (August 1988) 46: 29S-35S. Specific cyclophilins of interest as intracellular pharmacokinetic modulating proteins include cyclophilin A, B, C, D, E, and the like, where cyclophilin A is of particular interest.

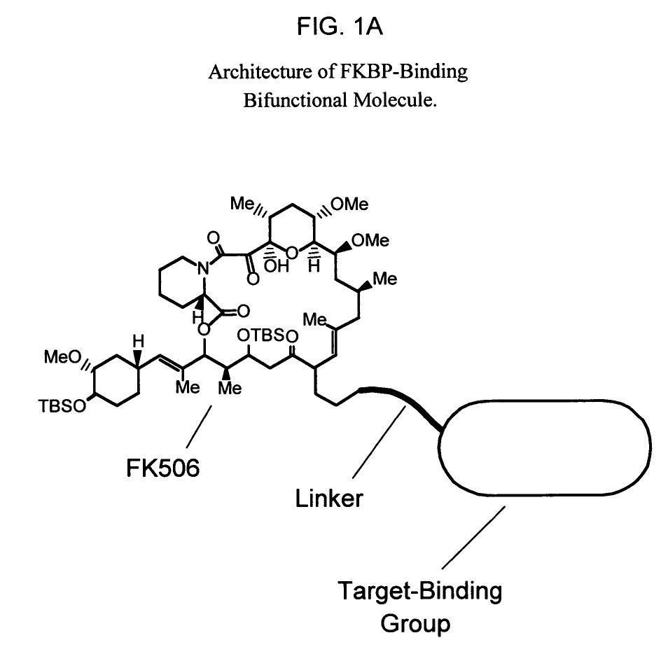

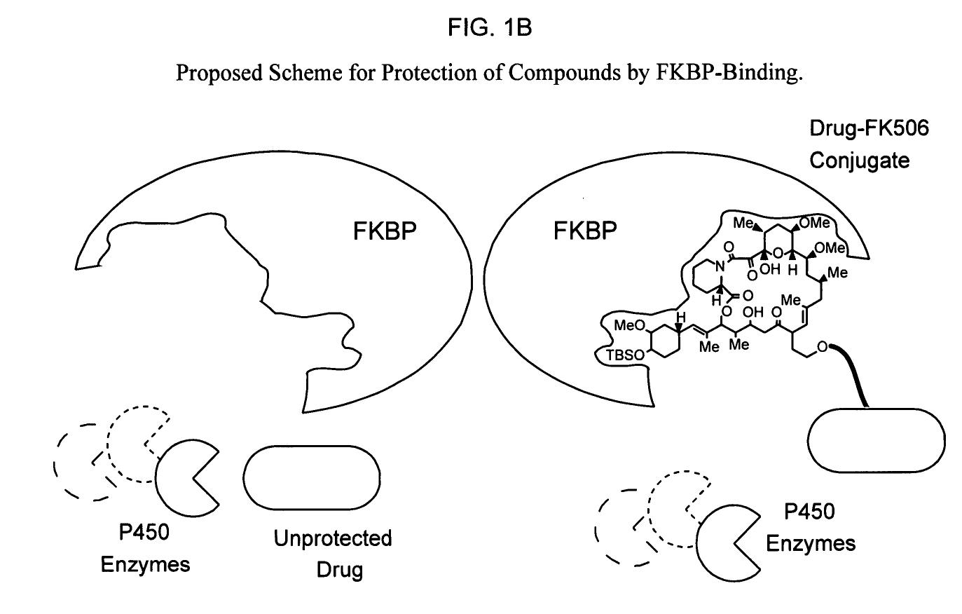

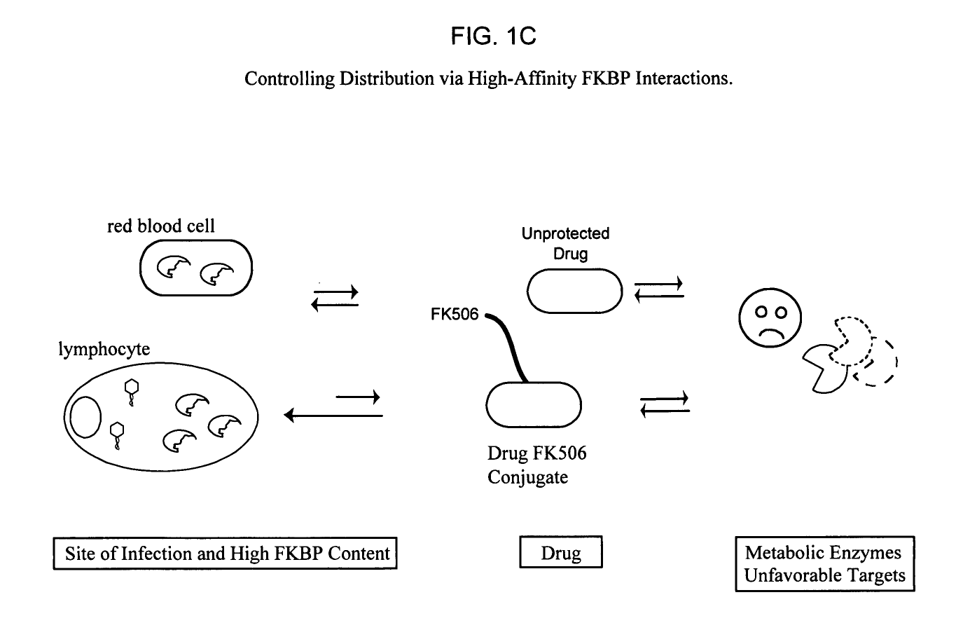

In general, the PMP can be included in the reaction mixture in a number of different ways. For example, the PMP can be added as a recombinantly expressed polypeptide. Alternatively, the PMP can be added as a composition comprising the PMP, such as cells expressing the PMP. For example, where the PMP is a peptidyl-prolyl isomerases, such as FKBP, the PMP can be added in the form of red blood cells or lymphocytes that include the peptidyl-prolyl isomerase (see FIG. 1B).

A representative assay is illustrated in FIGS. 1A to 1B. In the assay depicted in FIGS. 1A and 1B, a blocked fluoregenic substrate for a P450 metabolizer(s) is employed as the reporter, where this blocked fluorescent substrate competes with the drug of interest for being metabolized by the P450 enzyme(s). In the presence of a free drug, the metabolizers becomes active in metabolizing the free drug and more of the fluorescent substrate remains blocked, resulting in decreased fluorescence of the reaction as compared to the absence of the free drug. However, in the presence of a bifunctional molecule, the metabolizer does not metabolize the protected drug moiety and more of the fluorescent substrate is metabolized, resulting in increased fluorescence of the reaction as compared to a free drug control.

As such, in the presence of the PMP, the bifunctional compound (FIG. 1A) becomes associated with the PMP and as a result of the association the drug moiety of the bifunctional molecule is protected from the compound (FIG. 1B). Therefore, more of the reporter substrate will be metabolized by the enzyme. As a result, the detectable signal from the reporter substrate will increase. However, a free drug that is not linked a pharmacokinetic modulating moiety will not become associated with the PMP. Therefore, the free drug compete for being metabolized by the metabolizer with the reporter substrate. As a result, the detectable signal from the reporter substrate will decrease.

Once the components of the assay are combined to produce the reaction mixture, the reaction mixture is then evaluated for signal from the reporter to determine whether the bifunctional molecule has a modulated pharmacokinetic property as compared to a free drug control. The terms “assessing” and “evaluating” are used interchangeably to refer to any form of measurement, and includes determining if an element is present or not. The terms “determining,” “measuring,” and “assessing,” and “assaying” are used interchangeably and include both quantitative and qualitative determinations. Assessing may be relative or absolute. “Assessing the presence of” includes determining the amount of something present, as well as determining whether it is present or absent. The detectable signal from the reaction mixture can be evaluated at a single time point after the components of the reaction mixture are combined. Alternatively, the detectable signal from the reaction mixture can be evaluated at a plurality of time points over a period of time after the components of the reaction mixture are combined.

In some embodiments, the reaction mixture is evaluated at a single time point after the components of the reaction mixture are combined including, at least about 5 second to about 12 minutes, about 14 minutes, about 16 minutes, about 18 minutes, about 20 minutes or more, including about 30 minutes, about 35 minutes, about 40 minutes, about 45 minutes, about 50 minutes, about 55 minutes, about 1 hour or more after the components of the reaction mixture are combined. In some embodiments, the period of time for assaying detectable signal is at least about 5 seconds, about 10 second, about 15 second, about 20 seconds, about 25 seconds, about 30 seconds, about 35 seconds, about 40 seconds, about 45 seconds, about 50 seconds, about 55 seconds, about 60 seconds or more after the components of the reaction mixture are combined.

In other embodiments, once the components are combined the detectable signal of the reaction mixture is determined at a plurality of time points (e.g., time intervals) over a period of time ranging from about 5 second to about 20 minutes or more, including about 30 minutes, about 35 minutes, about 40 minutes, about 45 minutes, about 50 minutes, about 55 minutes, about 1 hour or more. In some embodiments, the period of time for assaying detectable signal is at least about 5 seconds, about 10 second, about 15 second, about 20 seconds, about 25 seconds, about 30 seconds, about 35 seconds, about 40 seconds, about 45 seconds, about 50 seconds, about 55 seconds, about 60 seconds or more, wherein the detectable signal of the reaction is measured at about 1 second intervals. For example, in embodiments in which the detectable signal is measured over a period of time of about 16 seconds, the fluorescence is measured at about the I second interval, at about the 2 second interval, at about the 3 second interval, and up to about the 16 second interval.

In other embodiments, the period of time for evaluating the detectable signal of the reaction mixture is at least about 2 minutes, about 4 minutes, about 6 minutes, about 8 minutes, about 10 minutes, about 12 minutes, about 14 minutes, about 16 minutes, about 18 minutes, about 20 minutes, about 22 minutes, about 24 minutes, about 26 minutes, about 28 minutes, about 30 minutes or more, wherein the fluorescence of the reaction is measures at about I minute intervals. For example, in embodiments in which the detectable signal is measured over a period of time of about 16 minutes, the detectable signal is measured at about the 1 minute interval, at about the 2 minute interval, at about the 3 minute interval, and up to about the 16 minute interval.

The above-described methods provide a measure of metabolizer activity in the reaction mixture, which in turn provides a measure of the amount of drug that is available for being metabolized by the metabolizer in the reaction mixture. The measure may be qualitative or quantitative, where quantitative refers to both relative and absolute quantitative determinations.

As such, the subject methods provide a way to readily determine whether a bifunctional molecule of a drug of interest exhibits improved activity as compared to its corresponding free drug control.

High-Throughout Assays and Devices

In certain embodiments, the subject methods are performed in a high throughput (HT) format. In the subject HT embodiments of the subject invention, a plurality of different compounds are simultaneously tested. By simultaneously tested is meant that each of the compounds in the plurality are tested at substantially the same time. Thus, at least some, if not all, of the compounds in the plurality are assayed for their effects in parallel. The number of compounds in the plurality of that are simultaneously tested is typically at least about 10, where in certain embodiments the number may be at least about 100 or at least about 1000, where the number of compounds tested may be higher. In general, the number of compounds that are tested simultaneously in the subject HT methods ranges from about 10 to 10,000, usually from about 100 to 10,000 and in certain embodiments from about 1000 to 5000. A variety of high throughput screening assays for determining the activity of candidate agent are known in the art and are readily adapted to the present invention, including those described in e.g., Schultz (1998) Bioorg Med Chem Lett 8:2409-2414; Weller (1997) Mol Divers. 3:61-70; Fernandes (1998) Curr Opin Chem Biol 2:597-603; Sittampalam (1997) Curr Opin Chem Biol 1:384-91; as well as those described in published United States application 20040072787 and issued U.S. Pat. No. 6,127,133; the disclosures of which are herein incorporated by reference.

As such, the subject assays can be run in a in a high-throughput manner with a plurality of candidate compounds (e.g., three or more, five or more, ten or more, 20 or more, 50, 100, 200, 250, 400, 500, 1000, or 10,000 or more, and the like) to screen for compounds having at least one modulated pharmacokinetic property as compared to a free drug control. The high-throughput assays of the invention can be especially useful in determining the spectrum of candidate compounds that have at least one modulated pharmacokinetic property as compared to a free drug control in the presence of a particular PMP. For example, plurality of candidate compounds can be added to a plurality of reaction mixtures all containing the same PMP. The detectable signals from the plurality of reaction mixtures can then be evaluated to determine whether the plurality of bifunctional compound has at least one modulated pharmacokinetic property as compared to the corresponding free drug controls.

The present invention also provides devices for high-throughput assays of the subject screening methods. As such, the device provides for screening a plurality of bifunctional molecules for a modulated pharmacokinetic property as compared to free drug controls. In some embodiments, the device will comprise an array of addressable reaction members, wherein each reaction member comprises a compound that metabolizes the drug, a reporter substrate of the compound, and a PMP. By “addressable reaction members” is meant a plurality (e.g., three or more, five or more, ten or more, 20 or more, 50, 100, 200, 250, 400, 500, 1000, or 10,000 or more, and the like) of reaction containers that are suitable for performing the subject screening assay. In certain embodiments, the plurality of addressable reaction members is a microtiter plate, typically having 6, 24, 96, 384 or even 1536 sample wells arranged in a rectangular matrix. In some embodiments, the device comprises a plurality of sample wells each comprising a cytochrome P450 (CYP), a fluorescent substrate; and a pharmacokinetic modulating protein (PMP).

Utility

The subject methods can be used to screen a variety of different types of candidate bifunctional molecules of drugs of interest. A candidate bifunctional molecule is a non-naturally occurring or synthetic compound that is a conjugate of a drug or derivative thereof and a pharmacokinetic modulating moiety, where these two moieties are optionally joined by a linking group. The targeted bifunctional molecule is further characterized in that the pharmacokinetic modulating and drug moieties are different, such that the bifunctional molecule may be viewed as a heterodimeric compound produced by the joining of two different moieties. In many embodiments, the pharmacokinetic modulating moiety and the drug moiety are chosen such that the corresponding drug target and binding partner of the pharmacokinetic modulating moiety, e.g. corresponding pharmacokinetic modulating protein to which the pharmacokinetic modulating moiety binds, do not naturally associate with each other to produce a biological effect. As indicated above, the subject bifunctional molecules are small. As such, the molecular weight of the bifunctional molecule is generally at least about 100 D, usually at least about 400 D and more usually at least about 500 D, and may be as great as 2000 D or greater, but usually does not exceed about 5000 D.

Bifunctional molecules of interest that are identified using the subject methods may be characterized in that they exhibit at least one modulated pharmacokinetic property, e.g. half-life, hepatic first-pass metabolism, volume of distribution, degree of albumin binding, etc., upon administration to a host as compared to a free drug control. By modulated pharmacokinetic property is meant that the bifunctional molecule exhibits a change with respect to at least one pharmacokinetic property as compared to a free drug control. For example, a bifunctional molecule of the subject invention may exhibit a modulated, e.g. longer, half-life than its corresponding free drug control. Similarly, a bifunctional molecule may exhibit a reduced propensity to be eliminated or metabolized upon its first pass through the liver as compared to a free drug control. Likewise, a given bifunctional molecule may exhibit a different volume of distribution that its corresponding free drug control, e.g. a higher amount of the bifunctional molecule may be found in the intracellular space as compared to a corresponding free drug control. Analogously, a given bifunctional molecule may exhibit a modulated degree of albumin binding such that the drug moiety's activity is not as reduced, if at all, upon binding to albumin as compared to its corresponding free drug control. In evaluating whether a given bifunctional molecule has at least one modulated pharmacokinetic property, as described above, the pharmacokinetic parameter of interest is typically assessed at a time at least 1 week, usually at least 3 days and more usually at least 1 day following administration, but preferably within about 6 hours and more preferably within about 1 hour following administration.

Bifunctional molecules of the subject invention are generally described by the formula:
Z-L-X
wherein:
X is a drug moiety;
L is bond or linking group; and
Z is pharmacokinetic modulating moiety;
with the proviso that X and Z are different.

Drug Moiety: X

The drug moiety X may be any molecule, as well as a binding portion or fragment, e.g. derivative, thereof, that is capable of modulating a biological process in a living host, either by itself or in the context of the pharmacokinetic modulating protein/bifunctional molecule binary complex. Generally, X is a small organic molecule that is capable of binding to the target of interest. As the drug moiety of the bifunctional molecule is a small molecule, it generally has a molecular weight of at least about 50 D, usually at least about 100 D, where the molecular weight may be as high as 500 D or higher, but will usually not exceed about 2000 D.

The drug moiety is capable of interacting with a target in the host into which the bifunctional molecule is administered during practice of the subject methods. The target may be a number of different types of naturally occurring structures, where targets of interest include both intracellular and extracellular targets, where such targets may be proteins, phospholipids, nucleic acids and the like, where proteins are of particular interest. Specific proteinaceous targets of interest include, without limitation, enzymes, e.g. kinases, phosphatases, reductases, cyclooxygenases, proteases and the like, targets comprising domains involved in protein-protein interactions, such as the SH2, SH3, PTB and PDZ domains, structural proteins, e.g. actin, tubulin, etc., membrane receptors, immunoglobulins, e.g. IgE, cell adhesion receptors, such as integrins, etc, ion channels, transmembrane pumps, transcription factors, signaling proteins, and the like.

The drug moiety of the bifunctional compound will include one or more functional groups necessary for structural interaction with the target, e.g. groups necessary for hydrophobic, hydrophilic, electrostatic or even covalent interactions, depending on the particular drug and its intended target. Where the target is a protein, the drug moiety will include functional groups necessary for structural interaction with proteins, such as hydrogen bonding, hydrophobic-hydrophobic interactions, electrostatic interactions, etc., and will typically include at least an amine, amide, sulfhydryl, carbonyl, hydroxyl or carboxyl group, preferably at least two of the functional chemical groups. As described in greater detail below, the drug moiety will also comprise a region that may be modified and/or participate in covalent linkage to the other components of the bifunctional molecule, such as the targeting moiety or linker, without substantially adversely affecting the moiety's ability to bind to its target.

The drug moieties often comprise cyclical carbon or heterocyclic structures and/or aromatic or polyaromatic structures substituted with one or more of the above functional groups. Also of interest as drug moieties are structures found among biomolecules, including peptides, saccharides, fatty acids, steroids, purines, pyrimidines, derivatives, structural analogs or combinations thereof. Such compounds may be screened to identify those of interest, where a variety of different screening protocols are known in the art.

The drug moiety of the bifunctional molecule may be derived from a naturally occurring or synthetic compound that may be obtained from a wide variety of sources, including libraries of synthetic or natural compounds. For example, numerous means are available for random and directed synthesis of a wide variety of organic compounds and biomolecules, including the preparation of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As such, the drug moiety may be obtained from a library of naturally occurring or synthetic molecules, including a library of compounds produced through combinatorial means, i.e. a compound diversity combinatorial library. When obtained from such libraries, the drug moiety employed will have demonstrated some desirable activity in an appropriate screening assay for the activity. Combinatorial libraries, as well as methods for the production and screening, are known in the art and described in: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

Specific drugs of interest from which the drug moiety may be derived include, but are not limited to: psychopharmacological agents, such as (1) central nervous system depressants, e.g. general anesthetics (barbiturates, benzodiazepines, steroids, cyclohexanone derivatives, and miscellaneous agents), sedative-hypnotics (benzodiazepines, barbiturates, piperidinediones and triones, quinazoline derivatives, carbamates, aldehydes and derivatives, amides, acyclic ureides, benzazepines and related drugs, phenothiazines, etc.), central voluntary muscle tone modifying drugs (anticonvulsants, such as hydantoins, barbiturates, oxazolidinediones, succinimides, acylureides, glutarimides, benzodiazepines, secondary and tertiary alcohols, dibenzazepine derivatives, valproic acid and derivatives, GABA analogs, etc.), analgesics (morphine and derivatives, oripavine derivatives, morphinan derivatives, phenylpiperidines, 2,6-methane-3-benzazocaine derivatives, diphenylpropylamines and isosteres, salicylates, p-aminophenol derivatives, 5-pyrazolone derivatives, arylacetic acid derivatives, fenamates and isosteres. etc.) and antiemetics (anticholinergics, antihistamines, antidopaminergics, etc.), (2) central nervous system stimulants, e.g. analeptics (respiratory stimulants, convulsant stimulants, psychomotor stimulants), narcotic antagonists (morphine derivatives, oripavine derivatives, 2,6-methane-3-benzoxacine derivatives. morphinan derivatives) nootropics, (3) psychopharmacologicals, e.g. anxiolytic sedatives (benzodiazepines, propanediol carbamates) antipsychotics (phenothiazine derivatives, thioxanthine derivatives, other tricyclic compounds, butyrophenone derivatives and isosteres, diphenylbutylamine derivatives, substituted benzamides, arylpiperazine derivatives, indole derivatives, etc.), antidepressants (tricyclic compounds, MAO inhibitors, etc.), (4) respiratory tract drugs, e.g. central antitussives (opium alkaloids and their derivatives);

pharmacodynamic agents, such as (1) peripheral nervous system drugs, e.g. local anesthetics (ester derivatives, amide derivatives), (2) drugs acting at synaptic or neuroeffector junctional sites, e.g. cholinergic agents, cholinergic blocking agents, neuromuscular blocking agents, adrenergic agents, antiadrenergic agents, (3) smooth muscle active drugs, e.g. spasmolytics (anticholinergics, musculotropic spasmolytics), vasodilators, smooth muscle stimulants, (4) histamines and antihistamines, e.g. histamine and derivative thereof (betazole), antihistamines (H₁-antagonists, H₂-antagonists), histamine metabolism drugs, (5) cardiovascular drugs, e.g. cardiotonics (plant extracts, butenolides, pentadienolids, alkaloids from erythrophleum species, ionophores, -adrenoceptor stimulants, etc), antiarrhythmic drugs, antihypertensive agents, antilipidemic agents (clofibric acid derivatives, nicotinic acid derivatives, hormones and analogs, antibiotics, salicylic acid and derivatives), antivaricose drugs, hemostyptics, (6) blood and hemopoietic system drugs, e.g. antianemia drugs, blood coagulation drugs (hemostatics, anticoagulants, antithrombotics, thrombolytics, blood proteins and their fractions), (7) gastrointestinal tract drugs, e.g. digestants (stomachics, choleretics), antiulcer drugs, antidiarrheal agents, (8) locally acting drugs;

chemotherapeutic agents, such as (1) anti-infective agents, e.g. ectoparasiticides (chlorinated hydrocarbons, pyrethins, sulfurated compounds). anthelmintics, antiprotozoal agents, antimalarial agents, antiamebic agents, antileiscmanial drugs, antitrichomonal agents, antitrypanosomal agents. sulfonamides, antimycobacterial drugs, antiviral chemotherapeutics, HIV protease inhibitors, such as amprenavir (AGENERASE), lopinavir (KELETRA), ritonavir, and (CRIXIVAN), etc., and (2) cytostatics, i.e. antineoplastic agents or cytotoxic drugs, such as alkylating agents, e.g. Mechlorethamine hydrochloride (Nitrogen Mustard, Mustargen, HN2), Cyclophosphamide (Cytovan, Endoxana), Ifosfamide (IFEX), Chlorambucil (Leukeran), Melphalan (Phenylalanine Mustard, L-sarcolysin, Alkeran, L-PAM), Busulfan (Myleran), Thiotepa (Triethylenethiophosphoramide), Carmustine (BICNU, BC-NU), Lomustine (CeeNU, CCNU), Streptozocin (Zanosar) and the like; plant alkaloids, e.g. Vincristine (Oncovin), Vinblastine (Velban, Velbe), Paclitaxel (Taxol), and the like; antimetabolites, e.g. Methotrexate (MTX), Mercaptopurine (Purinethol, 6-MP), Thioguanine (6-TG), Fluorouracil (5-FU), Cytarabine (Cytosar-U, Ara-C), Azacitidine (Mylosar, 5-AZA) and the like; antibiotics, e.g. Dactinomycin (Actinomycin D, Cosmegen), Doxorubicin (Adriamycin), Daunorubicin (duanomycin, Cerubidine), Idarubicin (Idamycin), Bleomycin (Blenoxane), Picamycin (Mithramycin, Mithracin), Mitomycin (Mutamycin) and the like, and other anticellular proliferative agents, e.g. Hydroxyurea (Hydrea), Procarbazine (Mutalane), Dacarbazine (DTIC-Dome), Cisplatin (Platinol) Carboplatin (Paraplatin), Asparaginase (Elspar) Etoposide (VePesid, VP-16-213), Amsarcrine (AMSA, m-AMSA), Mitotane (Lysodren), Mitoxantrone (Novatrone), and the like;

Antibiotics, such as: aminoglycosides, e.g. amikacin, apramycin, arbekacin, bambermycins, butirosin, dibekacin, dihydrostreptomycin, fortimicin, gentamicin, isepamicin, kanamycin, micronomcin, neomycin, netilmicin, paromycin, ribostamycin, sisomicin, spectinomycin, streptomycin, tobramycin, trospectomycin; amphenicols, e.g. azidamfenicol, chloramphenicol, florfenicol, and theimaphenicol; ansamycins, e.g. rifamide, rifampin, rifamycin, rifapentine, rifaximin; β-lactams, e.g. carbacephems, carbapenems, cephalosporins, cehpamycins, monobactams, oxaphems, penicillins; lincosamides, e.g. clinamycin, lincomycin; macrolides, e.g. clarithromycin, dirthromycin, erythromycin, etc.; polypeptides, e.g. amphomycin, bacitracin, capreomycin, etc.; tetracyclines, e.g. apicycline, chlortetracycline, clomocycline, etc.; synthetic antibacterial agents, such as 2,4-diaminopyrimidines, nitrofurans. quinolones and analogs thereof, sulfonamides, sulfones;

Antifungal agents, such as: polyenes, e.g. amphotericin B, candicidin, dermostatin, filipin, fungichromin, hachimycin, hamycin, lucensomycin, mepartricin, natamycin, nystatin, pecilocin, perimycin; synthetic antifungals, such as allylamines, e.g. butenafine, naftifine, terbinafme; imidazoles, e.g. bifonazole, butoconazole, chlordantoin, chlormidazole, etc., thiocarbamates, e.g. tolciclate, triazoles, e.g. fluconazole, itraconazole, terconazole;

Anthelmintics, such as: arecoline, aspidin, aspidinol, dichlorophene, embelin, kosin, napthalene, niclosamide, pelletierine, quinacrine, alantolactone, amocarzine, amoscanate, ascaridole, bephenium, bitoscanate, carbon tetrachloride, carvacrol, cyclobendazole, diethylcarbamazine, etc.;

Antimalarials, such as: acedapsone, amodiaquin, arteether, artemether, artemisinin, artesunate, atovaquone, bebeerine, berberine, chirata, chlorguanide, chloroquine, chlorprogaunil, cinchona, cinchonidine, cinchonine, cycloguanil, gentiopicrin, halofantrine, hydroxychloroquine, mefloquine hydrochloride, 3-methylarsacetin, pamaquine, plasmocid, primaquine, pyrimethamine, quinacrine, quinidine, quinine, quinocide, quinoline, dibasic sodium arsenate;

Antiprotozoan agents, such as: acranil, tinidazole, ipronidazole, ethylstibamine, pentamidine, acetarsone, aminitrozole, anisomycin, nifuratel, tinidazole, benzidazole, suramin, and the like.

Name brand drugs of interest include, but are not limited to: Rezulin™, Lovastatin™, Enalapril™, Prozac™, Prilosec™, Lipotor™, Claritin™, Zocor™, Ciprofloxacin™, Viagra™, Crixivan™, Ritalin™, and the like.

Drug compounds of interest from which drug moieties may be derived are also listed in: Goodman & Gilman's, The Pharmacological Basis of Therapeutics (9th Ed) (Goodman et al. eds) (McGraw-Hill) (1996); and 1999 Physician's Desk Reference (1998).

Specific compounds of interest also include, but are not limited to:

amyloid imaging agents, such as TZDM and as disclosed in U.S. Patent Publication no.'s 20040223912, 20040223909, 20030149250; curcuminoid compounds, such as such as curcumin, demethoxycurcumin, bisdemethoxycurcumin, tetrahydrocurcumin and the like.

antineoplastic agents, as disclosed in U.S. Pat. No.'s 5,880,161, 5,877,206, 5,786,344, 5,760,041, 5,753,668, 5,698,529, 5,684,004, 5,665,715, 5,654,484, 5,624,924, 5,618,813, 5,610,292, 5,597,831, 5,530,026, 5,525,633, 5,525,606, 5,512,678, 5,508,277, 5,463,181, 5,409,893, 5,358,952, 5,318,965, 5,223,503, 5,214,068, 5,196,424, 5,109,024, 5,106,996, 5,101,072, 5,077,404, 5,071,848, 5,066,493, 5,019,390, 4,996,229, 4,996,206, 4,970,318, 4,968,800, 4,962,114, 4,927,828, 4,892,887, 4,889,859, 4,886,790, 4,882,334, 4,882,333, 4,871,746, 4,863,955, 4,849,563, 4,845,216, 4,833,145, 4,824,955, 4,785,085, 476,925, 4,684,747, 4,618,685, 4,611,066, 4,550,187, 4,550,186, 4,544,501, 4,541,956, 4,532,327, 4,490,540, 4,399,283, 4,391,982, 4,383,994, 4,294,763, 4,283,394, 4,246,411, 4,214,089, 4,150,231, 4,147,798, 4,056,673, 4,029,661, 4,012,448;

psycopharmacological/psychotropic agents, as disclosed in U.S. Pat. No.'s 5,192,799, 5,036,070, 4,778,800, 4,753,951, 4,590,180, 4,690,930, 4,645,773, 4,427,694, 4,424,202, 4,440,781, 5,686,482, 5,478,828, 5,461,062, 5,387,593, 5,387,586, 5,256,664, 5,192,799, 5,120,733, 5,036,070, 4,977,167, 4,904,663, 4,788,188, 4,778,800, 4,753,951, 4,690,930, 4,645,773, 4,631,285, 4,617,314, 4,613,600, 4,590,180, 4,560,684, 4,548,938, 4,529,727, 4,459,306, 4,443,451, 4,440,781, 4,427,694, 4,424,202, 4,397,853, 4,358,451, 4,324,787, 4,314,081, 4,313,896, 4,294,828, 4,277,476, 4,267,328, 4,264,499, 4,231,930, 4,194,009, 4,188,388, 4,148,796, 4,128,717, 4,062,858, 4,031,226, 4,020,072, 4,018,895, 4,018,779, 4,013,672, 3,994,898, 3,968,125, 3,939,152, 3,928,356, 3,880,834, 3,668,210;

cardiovascular agents, as disclosed in U.S. Pat. No.'s 4,966,967, 5,661,129, 5,552,411, 5,332,737, 5,389,675, 5,198,449, 5,079,247, 4,966,967, 4,874,760, 4,954,526, 5,051,423, 4,888,335, 4,853,391, 4,906,634, 4,775,757, 4,727,072, 4,542,160, 4,522,949, 4,524,151, 4,525,479, 4,474,804, 4,520,026, 4,520,026, 5,869,478, 5,859,239, 5,837,702, 5,807,889, 5,731,322, 5,726,171, 5,723,457, 5,705,523, 5,696,111, 5,691,332, 5,679,672, 5,661,129, 5,654,294, 5,646,276, 5,637,586, 5,631,251, 5,612,370, 5,612,323, 5,574,037, 5,563,170, 5,552,411, 5,552,397, 5,547,966, 5,482,925, 5,457,118, 5,414,017, 5,414,013, 5,401,758, 5,393,771, 5,362,902, 5,332,737, 5,310,731, 5,260,444, 5,223,516, 5,217,958, 5,208,245, 5,202,330, 5,198,449, 5,189,036, 5,185,362, 5,140,031, 5,128,349, 5,116,861, 5,079,247, 5,070,099, 5,061,813, 5,055,466, 5,051,423, 5,036,065, 5,026,712, 5,011,931, 5,006,542, 4,981,843, 4,977,144, 4,971,984, 4,966,967, 4,959,383, 4,954,526, 4,952,692, 4,939,137, 4,906,634, 4,889,866, 4,888,335, 4,883,872, 4,883,811, 4,847,379, 4,835,157, 4,824,831, 4,780,538, 4,775,757, 4,774,239, 4,771,047, 4,769,371, 4,767,756, 4,762,837, 4,753,946, 4,752,616, 4,749,715, 4,738,978, 4,735,962, 4,734,426, 4,734,425, 4,734,424, 4,730,052, 4,727,072, 4,721,796, 4,707,550, 4,704,382, 4,703,120, 4,681,970, 4,681,882, 4,670,560, 4,670,453, 4,668,787, 4,663,337, 4,663,336, 4,661,506, 4,656,267, 4,656,185, 4,654,357, 4,654,356, 4,654,355, 4,654,335, 4,652,578, 4,652,576, 4,650,874, 4,650,797, 4,649,139, 4,647,585, 4,647,573, 4,647,565, 4,647,561, 4,645,836, 4,639,461, 4,638,012, 4,638,011, 4,632,931, 4,631,283, 4,628,095, 4,626,548, 4,614,825, 4,611,007, 4,611,006, 4,611,005, 4,609,671, 4,608,386, 4,607,049, 4,607,048, 4,595,692, 4,593,042, 4,593,029, 4,591,603, 4,588,743, 4,588,742, 4,588,741, 4,582,854, 4,575,512, 4,568,762, 4,560,698, 4,556,739, 4,556,675, 4,555,571, 4,555,570, 4,555,523, 4,550,120, 4,542,160, 4,542,157, 4,542 156, 4,542,155, 4,542 151, 4,537,981, 4,537,904, 4,536,514, 4,536,513, 4,533,673, 4,526,901, 4,526,900, 4,525,479, 4,524,151, 4,522,949, 4,521,539, 4,520,026, 4,517,188, 4,482 562, 4,474,804, 4,474,803, 4,472,411, 4,466,979, 4,463,015, 4,456,617, 4,456,616, 4,456,615, 4,418,076, 4,416,896, 4,252,815, 4,220,594, 4,190,587, 4,177,280, 4,164,586, 4,151,297, 4,145,443, 4,143,054, 4,123,550, 4,083,968, 4,076,834, 4,064,259, 4,064,258, 4,064,257, 4,058,620, 4,001,421, 3,993,639, 3,991,057, 3,982,010, 3,980,652, 3,968,117, 3,959,296, 3,951,950, 3,933,834, 3,925,369, 3,923,818, 3,898,210, 3,897,442, 3,897,441, 3,886,157, 3,883,540, 3,873,715, 3,867,383, 3,873,715, 3,867,383, 3,691,216, 3,624,126;

antimicrobial agents as disclosed in U.S. Pat. No.'s 5,902,594, 5,874,476, 5,874,436, 5,859,027, 5,856,320; 5,854,242, 5,811,091, 5,786,350, 5,783,177, 5,773,469, 5,762,919, 5,753,715, 5,741,526, 5,709,870, 5,707,990, 5,696,117, 5,684,042, 5,683,709, 5,656,591, 5,643,971, 5,643,950, 5,610,196, 5,608,056, 5,604,262, 5,595,742, 5,576,341, 5,554,373, 5,541,233, 5,534,546, 5,534,508, 5,514,715, 5,508,417, 5,464,832, 5,428,073, 5,428,016, 5,424,396, 5,399,553, 5,391,544, 5,385,902, 5,359,066, 5,356,803, 5,354,862, 5,346,913, 5,302,592, 5,288,693, 5,266,567, 5,254,685, 5,252,745, 5,209,930, 5,196,441, 5,190,961, 5,175,160, 5,157,051, 5,096,700, 5,093,342, 5,089,251, 5,073,570, 5,061,702, 5,037,809, 5,036,077, 5,010,109, 4,970,226, 4,916,156, 4,888,434, 4,870,093, 4,855,318, 4,784,991, 4,746,504, 4,686,221, 4,599 228, 4,552,882, 4,492,700, 4,489,098, 4,489,085, 4,487,776, 4,479,953, 4,477,448, 4,474,807, 4,470,994, 4,370,484, 4,337,199, 4,311,709, 4,308,283, 4,304,910, 4,260,634, 4,233,311, 4,215,131, 4,166,122, 4,141,981, 4,130,664, 4,089,977, 4,089,900, 4,069,341, 4,055,655, 4,049,665, 4,044,139, 4,002,775, 3,991,201, 3,966,968, 3,954,868, 3,936,393, 3,917,476, 3,915,889, 3,867,548, 3,865,748, 3,867,548, 3,865,748, 3,783,160, 3,764,676, 3,764,677;

anti-inflammatory agents as disclosed in U.S. Pat. No.'s 5,872,109, 5,837,735, 5,827,837, 5,821,250, 5,814,648, 5,780,026, 5,776,946, 5,760,002, 5,750,543, 5,741,798, 5,739,279, 5,733,939, 5,723,481, 5,716,967, 5,688,949, 5,686,488, 5,686,471, 5,686,434, 5,684,204, 5,684,041, 5,684,031, 5,684,002, 5,677,318, 5,674,891, 5,672,620 5,665,752, 5,656,661, 5,635,516, 5,631,283, 5,622,948, 5,618,835, 5,607,959, 5,593,980, 5,593,960, 5,580,888, 5,552,424, 5,552,422, 5,516,764, 5,510,361, 5,508,026, 5,500,417, 5,498,405, 5,494,927: 5,476,876 5,472,973 5,470,885, 5,470,842, 5,464,856, 5,464,849 5,462,952, 5,459,151, 5,451,686, 5,444,043 5,436,265; 5,432,181, RE034918, U.S. Pat. No. 5,393,756, 5,380,738, 5,376,670, 5,360,811, 5,354,768, 5,348,957, 5,347,029, 5,340,815, 5,338,753, 5,324,648, 5,319,099, 5,318,971, 5,312,821, 5,302,597, 5,298,633, 5,298,522, 5,298,498, 5,290,800, 5,290,788, 5,284,949, 5,280,045, 5,270,319, 5,266,562, 5,256,680, 5,250,700, 5,250,552, 5,248,682, 5,244,917, 5,240,929, 5,234,939, 5,234,937, 5,232,939, 5,225,571, 5,225,418, 5,220,025, 5,212,189, 5,212,172, 5,208,250, 5,204,365, 5,202,350, 5,196,431, 5,191,084, 5,187,175, 5,185,326, 5,183,906, 5,177,079, 5,171,864, 5,169,963, 5,155,122, 5,143,929, 5,143,928, 5,143,927, 5,124,455, 5,124,347, 5,114,958, 5,112,846, 5,104,656, 5,098,613, 5,095,037, 5,095,019, 5,086,064, 5,081,261, 5,081,147, 5,081,126, 5,075,330, 5,066,668, 5,059,602, 5,043,457, 5,037,835, 5,037,811, 5,036,088, 5,013,850, 5,013,751, 5,013,736, 500,654, 4,992,448, 4,992,447, 4,988,733, 4,988,728, 4,981,865, 4,962,119, 4,959,378, 4,954,519, 4,945,099, 4,942,236, 4,931,457, 4,927,835, 4,912,248, 4,910,192, 4,904,786, 4,904,685, 4,904,674, 4,904,671, 4,897,397, 4,895,953, 4,891,370, 4,870,210, 4,859,686, 4,857,644, 4,853,392, 4,851,412, 4,847,303, 4,847,290, 4,845,242, 4,835,166, 4,826,990, 4,803,216, 4,801,598, 4,791,129, 4,788,205, 4,778,818, 4,775,679, 4,772,703, 4,767,776, 4,764,525, 4,760,051, 4,748,153, 4,725,616, 4,721,712, 4,713,393, 4,708,966, 4,695,571, 4,686,235, 4,686,224, 4,680,298, 4,678,802, 4,652,564, 4,644,005, 4,632,923, 4,629,793, 4,614,741, 4,599,360, 4,596 828, 4,595,694, 4,595,686, 4,594,357, 4,585,755, 4,579,866, 4,578,390, 4,569,942, 4,567,201, 4,563,476, 4,559,348, 4,558,067, 4,556,672, 4,556,669, 4,539,326, 4,537,903, 4,536,503, 4,518,608, 4,514,415, 4,512,990, 4,501,755, 4,495,197, 4,493,839, 4,465,687, 4,440 779, 4,440,763, 4,435 420, 4,412,995, 4,400,534, 4,355,034, 4,335,141, 4,322,420, 4,275,064, 4,244,963, 4,235,908, 4,234,593, 4,226,887, 4,201,778, 4,181,720, 4,173,650, 4,173,634, 4,145,444, 4,128,664, 4,125,612, 4,124,726, 4,124,707, 4,117,135, 4,027,031, 4,024,284, 4,021,553, 4,021 550, 4,018,923, 4,012,527, 4,011,326, 3,998,970, 3,998,954, 3,993 763, 3,991,212, 3,984,405, 3,978,227, 3,978,219, 3,978,202, 3,975,543, 3,968,224, 3,959,368, 3,949,082, 3,949,081, 3,947,475, 3,936,450, 3,934,018, 3,930,005, 3,857,955, 3,856,962, 3,821,377, 3,821,401, 3,789,121, 3,789,123, 3,726,978, 3,694,471, 3,691,214, 3,678,169, 3,624,216;

immunosuppressive agents, as disclosed in U.S. Pat. No.'s 4,450,159, 4,450,159, 5,905,085, 5,883,119, 5,880,280, 5,877,184, 5,874,594, 5,843,452, 5,817,672, 5,817,661, 5,817,660, 5,801,193, 5,776,974, 5,763,478, 5,739,169, 5,723,466, 5,719,176, 5,696,156, 5,695,753, 5,693,648, 5,693,645, 5,691,346, 5,686 469, 5,686,424, 5,679,705, 5,679,640, 5,670,504, 5,665,774, 5,665,772, 5,648,376, 5,639,455, 5,633,277, 5,624,930, 5,622,970, 5,605,903, 5,604,229, 5,574,041, 5,565,560, 5,550,233, 5,545,734, 5,540,931, 5,532,248, 5,527,820, 5,516,797, 5,514,688, 5,512,687, 5,506,233, 5,506,228, 5,494,895, 5,484,788, 5,470,857, 5,464,615, 5,432,183, 5,431,896, 5,385,918, 5,349,061, 5,344,925, 5,330,993, 5,308,837, 5,290,783, 5,290,772, 5,284,877, 5,284,840, 5,273,979, 5,262,533, 5,260,300, 5,252,732, 5,250,678, 5,247,076, 5,244,896, 5,238,689, 5,219,884, 5,208,241, 5,208,228, 5,202,332, 5,192,773, 5,189,042, 5,169,851, 5,162,334, 5,151,413, 5,149,701, 5,147,877, 5,143,918, 5,138,051, 5,093,338, 5,091,389, 5,068,323, 5,068,247, 5,064,835, 5,061,728, 5,055,290, 4,981,792, 4,810,692, 4,410,696, 4,346,096, 4,342,769, 4,317,825, 4,256,766, 4,180,588, 4,000,275, 3,59,921;

analgesic agents, as disclosed in U.S. Pat No.'s 5,292,736, 5,688,825, 5,554,789, 5,455,230, 5,292,736, 5,298,522, 5,216,165, 5,438,064, 5,204,365, 5,017,578, 4,906,655, 4,906,655, 4,994,450, 4,749,792, 4,980,365, 4,794,110, 4,670,541, 4,737,493, 4,622,326, 4,536,512, 4,719,231, 4,533,671, 4,552,866, 4,539,312, 4,569,942, 4,681,879, 4,511,724, 4,556,672, 4,721,712, 4,474,806, 4,595,686, 4,440,779, 4,434,175, 4,608,374, 4,395,402, 4,400,534, 4,374,139, 4,361,583, 4,252,816, 4,251,530, 5,874,459, 5,688,825, 5,554,789, 5,455,230, 5,438,064, 5,298,522, 5,216,165, 5,204,365, 5,030,639, 5,017,578, 5,008,264, 4,994,450, 4,980,365, 4,906,655, 4,847,290, 4,844,907, 4,794,110, 4,791,129, 4,774,256, 4,749,792, 4,737,493, 4,721,712, 4,719,231, 4,681,879, 4,670,541, 4,667,039, 4,658,037, 4,634,708, 4,623,648, 4,622,326, 4,608,374, 4,595,686, 4,594,188, 4,569,942, 4,556,672, 4,552,866, 4,539,312, 4,536,512, 4,533,671, 4,511,724, 4,440,779, 4,434,175, 4,400,534, 4,395,402, 4,391,827, 4,374,139, 4,361,583, 4,322,420, 4,306,097, 4,252,816, 4,251,530, 4,244,955, 4,232,018, 4,209,520, 4,164,514 4,147,872, 4,133,819, 4,124,713, 4,117,012, 4,064,272, 4,022,836, 3,966,944;

cholinergic agents, as disclosed in U.S. Pat. No.'s 5,219,872, 5,219,873, 5,073,560, 5,073,560, 5,346,911, 5,424,301, 5,073,560, 5,219,872, 4,900,748, 4,786,648, 4,798,841, 4,782,071, 4,710,508, 5,482,938, 5,464,842, 5,378,723, 5,346,911, 5,318,978, 5,219,873, 5,219,872, 5,084,281, 5,073,560, 5,002,955, 4,988,710, 4,900,748, 4,798,841, 4,786,648, 4,782,071, 4,745,123, 4,710,508;

adrenergic agents, as disclosed in U.S. Pat. No.'s 5,091,528, 5,091,528, 4,835,157, 5,708,015, 5,594,027, 5,580,892, 5,576,332, 5,510,376, 5,482,961, 5,334,601, 5,202,347, 5,135,926, 5,116,867, 5,091,528, 5,017,618, 4,835,157, 4,829,086, 4,579,867, 4,568,679, 4,469,690, 4,395,559, 4,381,309, 4,363,808, 4,343,800, 4,329,289, 4,314,943, 4,311,708, 4,304,721, 4,296,117, 4,285,873, 4,281,189, 4,278,608, 4,247,710, 4,145,550, 4,145,425, 4,139,535, 4,082,843, 4,011,321, 4,001,421, 3,982,010, 3,940,407, 3,852,468, 3832470;

antihistamine agents, as disclosed in U.S. Pat. No.'s 5,874,479, 5,863,938, 5,856,364, 5,770,612, 5,702,688, 5,674,912, 5,663,208, 5,658,957, 5,652,274, 5,648,380, 5,646,190, 5,641,814, 5,633,285, 5,614,561, 5,602,183, 4,923,892, 4,782,058, 4,393,210, 4,180,583, 3,965,257, 3,946,022, 3,931,197;

steroidal agents, as disclosed in U.S. Pat. No.'s 5,863,538, 5,855,907, 5,855,866, 5,780,592, 5,776,427, 5,651,987, 5,346,887, 5,256,408, 5,252,319, 5,209,926, 4,996,335, 4,927,807, 4,910,192, 4,710,495, 4,049,805, 4,004,005, 3,670,079, 3,608,076, 5,892,028, 5,888,995, 5,883,087, 5,880,115, 5,869,475, 5,866,558, 5,861,390, 5,861,388, 5,854,235, 5,837,698, 5,834,452, 5,830,886, 5,792,758, 5,792,757, 5,763,361, 5,744,462, 5,741,787, 5,741,786, 5,733,899, 5,731,345, 5,723,638, 5,721,226, 5,712,264, 5,712,263, 5,710,144, 5,707,984, 5,705,494, 5,700,793, 5,698,720, 5,698,545, 5,696,106, 5,677,293, 5,674,861, 5,661,141, 5,656,621, 5,646,136, 5,637,691, 5,616,574, 5,614,514, 5,604,215, 5,604,213, 5,599,807, 5,585,482, 5,565,588, 5,563,259, 5,563,131, 5,561,124, 5,556,845, 5,547,949, 5,536,714, 5,527,806, 5,506,354, 5,506,221, 5,494,907, 5,491,136, 5,478,956, 5,426,179, 5,422,262, 5,391,776, 5,382,661, 5,380,841, 5,380,840, 5,380,839, 5,373,095, 5,371,078, 5,352,809, 5,344,827, 5,344,826, 5,338,837, 5,336,686, 5,292,906, 5,292,878, 5,281,587, 5,272,140, 5,244,886, 5,236,912, 5,232,915, 5,219,879, 5,218,109, 5,215,972, 5,212,166, 5,206,415, 5,194,602, 5,166,201, 5,166,055, 5,126,488, 5,116,829, 5,108,996, 5,099,037, 5,096,892, 5,093,502, 5,086,047, 5,084,450, 5,082,835, 5,081,114, 5,053,404, 5,041,433, 5,041,432, 5,034,548, 5,032,586, 5,026,882, 4,996,335, 4,975,537, 4,970,205, 4,954,446, 4,950,428, 4,946,834, 4,937,237, 4,921,846, 4,920,099, 4,910,226, 4,900,725, 4,892,867, 4,888,336, 4,885,280, 4,882,322, 4,882,319, 4,882,315, 4,874,855, 4,868,167, 4,865,767, 4,861,875, 4,861,765, 4,861,763, 4,847,014, 4,774,236, 4,753,932, 4,711,856, 4,710,495, 4,701,450, 4,701,449, 4,689,410, 4,680,290, 4,670,551, 4,664,850, 4,659,516, 4,647,410, 4,634,695, 4,634,693, 4,588,530, 4,567,000, 4,56,0557, 4,558,041, 4,552,871, 4,552,868, 4,541,956, 4,519,946, 4,515,787, 4,512,986, 4,502,989, 4,495,102; the disclosures of which are herein incorporated by reference.

The drug moiety of the bifunctional molecule may be the whole compound or a derivative thereof, e.g. a binding fragment or portion thereof, that retains its affinity and specificity for the target of interest, and therefore its desired activity, while having a linkage site for covalent bonding to the targeting moiety or linker.

Pharmacokinetic Modulating Moiety: Z

Z is a pharmacokinetic modulating moiety that is a ligand for a biological entity endogenous to the host to which the bifunctional molecule is administered, where binding of the modulating moiety to this biological entity results in modulation of at least one pharmacokinetic property of the drug moiety of the bifunctional molecule, as compared to the drug moiety's corresponding free drug control. In many embodiments, this biological entity to which the modulating moiety of the bifunctional molecule binds is a protein, e.g. an intracellular or extracellular protein. As such, in many embodiments this biological entity is properly referred to the endogenous pharmacokinetic modulating protein.

The binding interaction between the modulating moiety of the bifunctional molecule and the endogenous biological entity, e.g. endogenous pharmacokinetic modulating protein, is non-covalent, such that no covalent bonds are produced between the bifunctional molecule and the pharmacokinetic modulating protein upon binding of the two entities. As the pharmacokinetic modulating moiety of the bifunctional molecule is a small molecule, it generally has a molecular weight of at least about 50 D, usually at least about 100 D, where the molecular weight may be as high as 500 D or higher, but will usually not exceed about 2000 D. In certain embodiments, the pharmacokinetic modulating moiety, in the context of the bifunctional molecule, has substantially no pharmacological activity at its effective concentration beyond binding to its corresponding endogenous pharmacokinetic modulating protein, i.e. it does not directly cause a pharmacokinetic modulating protein-mediated pharmacological event to occur upon binding at its effective concentration to the pharmacokinetic modulating protein, where a pharmacokinetic modulating protein mediated pharmacological event is a pharmacologically relevant event which is directly modulated by the pharmacokinetic modulating protein in the absence of the subject bifunctional molecules. In other certain embodiments, the modulating moiety may have some pharmacological activity, where this pharmacological activity does not adversely effect the host to the extent that the therapy in which the bifunctional molecule is employed places the host in a worst condition than prior to the therapy. In other words, pharmacological activity in the modulating moiety may be tolerated in these embodiments to the extent that any consequences of such activity, if any, are outweighed by the benefits provided by the bifunctional molecule. As used herein, pharmacological event is an event that is distinct from a biochemical event (e.g. inhibition a prolyl isomerase activity) or a biological event (e.g. inducement of a cell to express new genes).

The pharmacokinetic modulating protein to which the modulating moiety of the bifunctional molecule binds may be any protein that is present in the host at the time the bifunctional molecule is introduced to the host, i.e. the pharmacokinetic modulating protein is one that is endogenous to the host. The pharmacokinetic modulating protein may or may not have one or more modified residues, e.g. residues that are glycosylated, such that it may or may not be a glycoprotein. Furthermore, the pharmacokinetic modulating protein to which the bifunctional molecule binds via the pharmacokinetic modulating moiety may or may not be part of a complex or structure of a plurality of biological molecules, e.g. lipids, where such complexes or structures may include lipoproteins, lipid bilayers, and the like. However, in many embodiments, the pharmacokinetic modulating protein to which the bifunctional molecule binds will be by itself, i.e. will not be part of a larger structure of a plurality of biological molecules. Though the pharmacokinetic modulating protein may be a protein that is not native to the host but has been introduced at some time prior to introduction of the bifunctional molecule, e.g. through prior administration of the protein or a nucleic acid composition encoding the same, such as through gene therapy, the pharmacokinetic modulating protein will, in many embodiments, be a protein that is native to and naturally expressed by at least some of the host's cells, i.e. a naturally occurring protein in the host. The pharmacokinetic modulating protein is a protein that is present in the region of host occupied by the drug target. As such, where the drug target is an intracellular drug target, the pharmacokinetic protein will be an intracellular protein present in the cell comprising the target, typically expressed in the cell comprising the target, i.e. the pharmacokinetic modulating protein and target are co-expressed in the same cell. Likewise, where the drug target is an extracellular drug target, the pharmacokinetic modulating protein will be an extracellular protein that is found in the vicinity of the target.

Although not a requirement in certain embodiments, in many preferred embodiments the pharmacokinetic modulating protein is one that is present in the host in sufficient quantities such that, upon binding of at least a portion of pharmacokinetic modulating protein present in the host to the bifunctional molecule, adverse pharmacological effects do not occur. In other words, the pharmacokinetic modulating protein in these preferred embodiments is one in which its native and desirable biological activity, if any, is not diminished by an unacceptable amount following binding of the portion of the pharmacokinetic modulating protein population to the bifunctional molecule. The amount of diminished activity of the pharmacokinetic modulating protein that is acceptable in a given situation is determined with respect to the condition being treated in view of the benefits of treatment versus the reduction of overall pharmacokinetic modulating protein activity, if any. In certain situations, a large decrease in overall pharmacokinetic modulating protein activity may be acceptable, e.g. where the pharmacokinetic modulating protein activity aggravates the condition being treated.

The specific pharmacokinetic modulating protein to which the modulating moiety of the subject bifunctional molecule binds may vary greatly depending on the desired modulation of the one or more pharmacokinetic properties or parameters of interest. For example, where one wishes to modulated the half-life, hepatic first-pass metabolism, or volume of distribution, intracellular proteins are often of interest, where representative intracellular proteins of interest include: peptidyl-prolyl isomerases, e.g. FKBPs and cyclophilins; ubiquitously expressed molecular chaperones, e.g. Heat Shock Protein 90 (Hsp90); steroid hormone receptors, e.g. estrogen receptors, glucocorticoid receptors, androgen receptors; retinoic acid binding protein, cytoskeletal proteins, such as tubulin and actin; etc. Of particular interest as intracellular pharmacokinetic modulating proteins are cis-trans peptidyl-prolyl isomerases which interact with many proteins because of their chaperonin/isomerase activity, e.g. FKBPs and cyclophilins. Peptidyl-prolyl isomerases of interest include FKBPs. A number of different FKBPs are known in the art, and include those described in: Sabatini et al., Mol. Neurobiol. (October 1997) 15:223-239; Marks, Physiol. Rev. (July 1996) 76:631-649; Kay, Biochem J. (March, 1996) 314: 361-385; Braun et al., FASEB J. (January 1995) 9:63-72; Fruman et al, FASEB J. (April 1994) 8:391-400; and Hacker et al., Mol. Microbiol. (November 1993) 10: 445-456. FKBPs of interest include FKBP 12, FKBP 52, FKBP 14.6 (described in U.S. Patent No. 5,525,523, the disclosure of which is herein incorporated by reference); FKBP 12.6 (described in U.S. Pat. No. 5,457,182 the disclosure of which is herein incorporated by reference); FKBP 13 (described in U.S. Pat. No.5,498,597, the disclosure of which is herein incorporated by reference); and HCB (described in U.S. Pat. No. 5,196,352 the disclosure of which is herein incorporated by reference); where FKBP 12 and FKBP 52 are of particular interest as intracellular pharmacokinetic modulating proteins. Also of specific interest as intracellular pharmacokinetic modulating proteins are cyclophilins. A number of cyclophilins are known in the art and are described in Trandinh et al., FASEB J. (December 1992) 6: 3410-3420; Harding et al., Transplantation (August 1988) 46: 29S-35S. Specific cyclophilins of interest as intracellular pharmacokinetic modulating proteins include cyclophilin A, B, C, D, E, and the like, where cyclophilin A is of particular interest.

Instead of being an intracellular protein, in certain embodiments the endogenous pharmacokinetic modulating protein is an extracellular or serum protein, e.g. where extracellular pharmacokinetic modulating proteins find use in the modulating of half-life, volume of distribution, and degree of albumin binding or albumin binding effect. Serum pharmacokinetic modulating proteins of particular interest are those that are relatively abundant in the serum of the host and meet the above criteria for suitable endogenous pharmacokinetic modulating proteins. By relatively abundant is meant that the concentration of the serum pharmacokinetic modulating protein is at least about 1 ng/ml, usually at least about 10 μg/ml and more usually at least about 15 μg/ml. Specific serum proteins of interest as pharmacokinetic modulating proteins include: albumin, Vitamin A binding proteins and Vitamin D binding proteins, β-2 macroglobulin, α-1 acid glycoprotein, with albumin being a particularly preferred pharmacokinetic modulating protein in many embodiments.

The Z moiety of the subject bifunctional molecules will therefore be chosen in view of the endogenous pharmacokinetic modulating protein that is to be used to bind to the bifunctional molecule and thereby achieve the desired pharmacokinetic property modulation. As such, the Z moiety may be a number of different ligands, depending on the particular endogenous pharmacokinetic modulating protein to which it is intended to bind. In many preferred embodiments, the Z moiety has an affinity for its pharmacokinetic modulating protein of at least about 10⁻⁴ M, usually at least about 10⁻⁶ molar and more usually at least about 10⁻⁸ M, where in many embodiments the Z moiety has an affinity for its pharmacokinetic modulating protein of between about 10⁻⁹ M and 10⁻¹² M. The Z moiety portion of the bifunctional molecule should also be specific for the pharmacokinetic modulating protein in the context of its binding activity when present in the bifunctional molecule, in that it does not significantly bind or substantially affect non-pharmacokinetic modulating proteins when it is present in the bifunctional molecule.

Representative ligands capable of serving as the Z moiety of the bifunctional molecule include ligands for intracellular proteins, such as: peptidyl-prolyl isomerase ligands, e.g. FK506, rapamycin, cyclosporin A and the like; Hsp90 ligands, e.g. geldanamycin; steroid hormone receptor ligands, e.g. naturally occurring steroid hormones, such as estrogen, progestin, testosterone, and the like, as well as synthetic derivatives and mimetics thereof, particularly those which bind with high specificity and affinity but do not activate their respective receptors; small molecules that bind to cytoskeletal proteins, e.g. antimitotic agents, such as taxanes, colchicine, colcemid, nocadozole, vinblastine, and vincristine, actin binding agents, such as cytochalasin, latrunculin, phalloidin, and the like.

As mentioned above, in certain preferred embodiments the intracellular pharmacokinetic modulating proteins are members of the peptidyl-prolyl isomerase family, particularly the FKBP and cyclophilin subsets of this family. Where peptidyl-prolyl isomerase pharmacokinetic modulating proteins are employed, the bifunctional molecule/peptidyl-prolyl isomerase complex will preferably not substantially bind to the natural peptidyl-prolyl isomerase/ligand target calcineurin so as to result in significant immunosuppression. A variety of ligands are known that bind to FKBPs and may be used in the subject invention. The ligands should specifically bind to an FKBP and have an affinity for the FKBP that is between about 10⁻⁶ M and about 10⁻¹⁰ M. Of interest are both naturally occurring FKBP ligands, including FK506 and rapamycin. Also of interest are synthetic FKBP ligands, including those described in U.S. Pat. Nos. 5,665,774; 5,622,970; 5,516,797; 5,614,547; and 5,403,833, the disclosures of which are herein incorporated by reference.

Also of interest in this particular set of preferred embodiments are cyclophilin ligands, where such ligands should specifically bind to cyclophilin with an affinity that is between about 10⁻⁶ M and about 10⁻⁹ M, including about 10⁻⁷M, and about 10⁻⁸M. A variety of ligands that bind to cyclophilins are also known, where such ligands include the naturally occurring cyclosporins, such as cyclosporin A, as well as synthetic derivatives and mimetics thereof, including those described in U.S. Pat. Nos.: 5,401,649; 5,318,901; 5,236,899; 5,227,467; 5,214,130; 5,122,511; 5,116,816; 5,089,390; 5,079,341; 5,017,597; 4,940,719; 4,914,188; 4,885,276; 4,798,823; 4,771,122; 4,703,033; 4,554,351; 4,396,542; 4,289,851; 4,288,431; 4,220,61 and 4,210,581, the disclosures of which are herein incorporated by reference.

Representative ligands for use as the Z moiety in the bifunctional molecule also include ligands that bind to extracellular pharmacokinetic modulating proteins. Such ligands should specifically bind to their respective extracellular pharmacokinetic modulating protein with an affinity of at least about 10⁻⁴ M or more, including an affinity of at least 10⁻⁵ M or more, at least 10⁻⁶ M, at least 10⁻⁷ M or more. Ligands of interest for use in binding to extracellular pharmacokinetic modulating proteins include: albumin ligands, such as arachidonate, bilirubin, hemin, aspirin, ibuprofen, para-amino salicylic acid, myristylate, plamitate, linoleate, warfarin, sulfisoxazole, chlorpromazine, etc.; α-l acid glycoprotein ligands, e.g. small neutral or basic molecules, e.g. propanolol, chlorpromazine, dipyrimadole, metoclopramide, aprindine, verapamil, and the like; Vitamin A and derivatives thereof, Vitamin D and derivatives thereof, and the like.

Linking Moiety: L

The Z and X moieties of the bifunctional molecule are joined together optionally through linking moiety L, where L may be either a bond or a linking group. Where linking groups are employed, such groups are chosen to provide for covalent attachment of the drug and ligand moieties through the linking group, as well as the desired structural relationship of the bifunctional molecule with respect to its intended pharmacokinetic modulating protein. Linking groups of interest may vary widely depending on the nature of the drug and ligand moieties. The linking group, when present, should preferably be biologically inert. Appropriate linkers can readily be identified using the affinity, specificity or selectivity assays described supra.

A variety of linking groups are known to those of skill in the art and find use in the subject bifunctional molecules. The linker groups should be sufficiently small so as to provide a bifunctional molecule having the overall size characteristics as described above, the size of the linker group, when present, is generally at least about 50 daltons, usually at least about 100 daltons and may be as large as 1000 daltons or larger, but generally will not exceed about 500 daltons and usually will not exceed about 300 daltons.

Generally, such linkers will comprise a spacer group terminated at either end with a reactive functionality capable of covalently bonding to the drug or ligand moieties. Spacer groups of interest include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex.

Specific spacer elements include: 1,4-diaminohexane, xylylenediamine, terephthalic acid, 3,6-dioxaoctanedioic acid, ethylenediamine-N,N-diacetic acid, 1,1′-ethylenebis(5-oxo-3-pyrrolidinecarboxylic acid), 4,4′-ethylenedipiperidine. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides. Specific linker groups that may find use in the subject bifunctional molecules include heterofunctional compounds, such as aziobenzoyl hydrazide, N-[4-(p-azidosalicylamino)butyl]-3′-[2′-pyridydithio]propionamid), bis-sulfosuccinimidyl suberate, dimethyladipimidate, disuccinimidyltartrate, N- -maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl 4-[N-maleimidomethyl]cyclohexane-1-carboxylate, 3-(2-pyridyldithio)propionic acid N-hydroxysuccinimide ester (SPDP), 4-(N-maleimidomethyl)-cyclohexane-1-carboxylic acid N-hydroxysuccinimide ester (SMCC), and the like.

The candidate bifunctional molecules may be prepared using any convenient methodology. In many embodiments of the subject invention, the invention is used to modulate the pharmacokinetic properties of an identified and at least partially characterized small molecule drug. Generally, a small molecule drug of interest is first identified. The drug may be a previously identified biologically active agent or compound having the desired target binding activity, or one that has been newly discovered using one or more drug discovery techniques. The bifunctional molecule is then generally produced from the drug using a rational or combinatorial approach.

In a rational approach, the bifunctional molecules are constructed from their individual components, e.g. pharmacokinetic modulating moiety, linker and drug. The components can be covalently bonded to one another through functional groups, as is known in the art, where such functional groups may be present on the components or introduced onto the components using one or more steps, e.g. oxidation reactions, reduction reactions, cleavage reactions and the like. Functional groups that may be used in covalently bonding the components together to produce the bifunctional molecule include: hydroxy, sulfhydryl, amino, and the like. The particular portion of the different components that are modified to provide for covalent linkage will be chosen so as not to substantially adversely interfere with that components desired binding activity, e.g. for the drug moiety, a region that does not affect the target binding activity will be modified, such that a sufficient amount of the desired drug activity is preserved. Where necessary and/or desired, certain moieties on the components may be protected using blocking groups, as is known in the art, see, e.g. Green & Wuts, Protective Groups in Organic Synthesis (John Wiley & Sons) (1991).

The above component approach to production of the bifunctional molecule is best suited for situations where the crystal structures of the pharmacokinetic modulating protein, the pharmacokinetic modulating moiety, the drug and the target are known, such that molecular modeling can be used to determine the optimal linker size, if any, to be employed to join the different components.

Alternatively, the bifunctional molecule can be produced using combinatorial methods to produce large libraries of potential bifunctional molecules which may then be screened for identification of a bifunctional molecule with the pharmacokinetic profile. Methods for producing and screening combinatorial libraries of molecules include: U.S. Pat. Nos. 5,741,713; 5,734,018; 5,731,423; 5,721,099; 5,708,153; 5,698,673; 5,688,997; 5,688,696; 5,684,711; 5,641,862; 5,639,603; 5,593,853; 5,574,656; 5,571,698; 5,565,324; 5,549,974; 5,545,568; 5,541,061; 5,525,735; 5,463,564; 5,440,016; 5,438,119; 5,223,409, the disclosures of which are herein incorporated by reference.

Alternatively, the bifunctional molecule may be produced using medicinal chemistry and known structure-activity relationships for the targeting moiety and the drug. In particular, this approach will provide insight as to where to join the two moieties to the linker.

As mentioned above, one class of exemplary bifunctional molecules comprise a pharmacokinetic modulating moiety that binds to an intracellular protein. In these embodiments, of particular interest are those bifunctional molecules in which the pharmacokinetic modulating moiety specifically binds to endogenous peptidyl-prolyl isomerase pharmacokinetic modulating proteins present in the host into which the bifunctional molecule is introduced. Thus, bifunctional molecules of interest include those in which the endogenous pharmacokinetic modulating protein is either an FKBP or a cyclophilin.

In preparing bifunctional molecules from FK506, a suitable attachment site on the FK506 structure is identified, modified as necessary, and then covalently attached to the linker or drug moiety. The structure of FK506 (also known as tacrolimus) is:

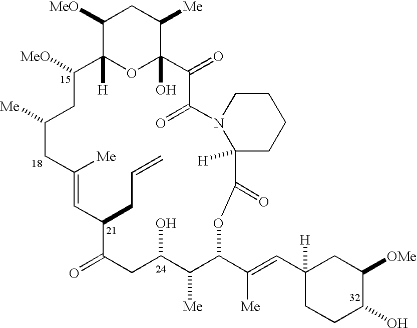

The site to which the linker/drug moiety is covalently attached is one that, upon covalent attachment, does not ablate the affinity and/or specificity of FK506 for its FKBP pharmacokinetic modulating protein, e.g. FKBP 12 or FKBP 52. As such, positions suitable for use as covalent linkage sites include atoms located between carbon 15 and carbon 25 and the substituents attached to these atoms. For example, oxidation of the allyl group or oxidation of the carbon 18 methylene group; modification of the carbon 22 ketone or the carbon 24 hydroxyl group or alkylation at carbon 21 or carbon 23; as well as the secondary hydroxyl group located on the cyclohexyl ring (carbon 32); are potential specific covalent linkage sites.

With FK506. depending on the drug moiety and/or linker to be attached, it may be desirable to introduce one or more functional moieties onto the FK506 structure. Functional moieties of interest that may be introduced include: hydroxyl groups, amino groups, carboxyl groups, aldehydes, carbonates, carbamates, azides, thiols, and esters, etc. Such groups may be introduced using known protocols, such as oxidation reactions, reduction reactions, cleavage reactions and the like, with or without the use of one or more blocking groups to prevent unwanted side reactions.

In some instances, it is desirable to covalently attach the drug moiety directly to FK506, often activated FK506. In such instances, the reactive functional group(s) introduced onto the FK506 structure will depend primarily on the nature of the drug moiety to be attached. Thus, for peptidic drug moieties, specific pairings of interest include: FK506 carbonates for reacting with amino groups of peptides; FK506 carboxylic acids for reacting with amino groups of peptides; FK506 amines for reacting with carboxylic acid groups of peptides; FK506 maleimide for reacting with thiol groups of peptides; and the like. Alternatively, where the drug moiety is a steroid, potential pairings of interest include: FK506 N-hydroxysuccinimidyl carbonate and partner amine; FK506 aldehyde and partner amine; FK506 aldehyde and partner hydrazide; FK506 hydroxy group and partner carboxylic acid OR alkyl halide; FK506 thiol and partner maleimide and the like.

Following introduction of the reactive functional group(s) onto the FK506 structure, the activated FK506 is then combined with the drug moiety/linker under conditions sufficient for covalent bonding to occur.

Another embodiment of particular interest are bifunctional molecules of cyclosporin A or analogs thereof. The structure of cyclosporin A is:

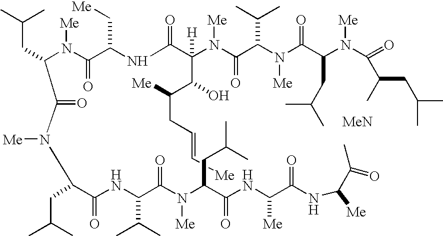

As with the FK506 bifunctional molecules, cyclosporin A will be conjugated to the drug moiety in a manner such that cyclosporin A does not substantially lose its affinity for cyclophilin. Preferred positions on the cyclosporin A structure that may serve as suitable covalent linkage sites include: residues 4, 5, 6, 7, 8; while less preferred but still possible residues include: 1,2, 3, 9, 10 and 11. Where necessary, reactive functionalities may be introduced onto the cyclosporin structure, where such functionalities include: hydroxyl groups, amino groups, carboxyl groups, aldehydes, carbonates, carbamates, azides, thiols, and esters, etc., with the particular functionality of interest being chosen with respect to the specific linker or drug moiety to be attached.

As mentioned above, the subject methods find use in identifying bifunctional molecules that exhibit at least one modulated pharmacokinetic property upon administration to a host as compared to their corresponding free drug, i.e. a free drug control. In other words, at least one of the pharmacokinetic properties of the subject bifunctional molecules differ from that of the corresponding free drug. Specific pharmacokinetic properties that may differ in the subject bifunctional molecules are drug half-life, drug first-pass metabolism, drug volume of distribution and degree of drug binding to a serum protein, e.g. albumin. In certain embodiments, the above improvements are achieved through the formation of binary or tripartite complexes, as described in application Ser. No. 09/316,932 entitled Bifunctional Molecules and Therapies Based Thereon, the disclosure of which is herein incorporated by reference.

In embodiments where the half-life of a drug is modulated, e.g. prolonged, by incorporating it into a bifunctional molecule, the modulating moiety of the bifunctional molecule may be a ligand for an intracellular or extracellular protein, as described above. In such embodiments, the modulating moiety is a ligand for an intracellular or extracellular protein that will serve to, when bound to the bifunctional molecule, i.e. when present as a binary complex with the bifunctional molecule, protect or shield the drug moiety from metabolism, biotransformation or excretion, e.g. by the liver or the kidney. These intracellular pharmacokinetic modulating proteins are generally proteins that are highly abundant in cells and thus the intracellular space of the host, e.g. the interior or erythrocytes, etc. These proteins serve as a protective reservoir for the bifunctional molecule and drug component thereof, thereby extending the half-life of the drug. Representative intracellular proteins for which the modulating moiety may serve as a ligand in this embodiment include the peptidyl prolyl isomerases, heat shock proteins (hsp's), tubulins, and the like, where additional potential intracellular pharmacokinetic modulating proteins are described supra. Extracellular pharmacokinetic modulating proteins are also of interest, where the proteins should be long-lived proteins that impart their long half-life to the bifunctional molecule and the drug component thereof upon formation of a binary complex with the bifunctional molecule. Representative extracellular pharmacokinetic modulating proteins of interest include: albumin, α1-acid glycoprotein, and the like. In the above embodiments, the half-life the drug component of the bifunctional molecule is generally prolonged as compared to the corresponding free drug control by a factor of at least about 1.5, including at least a factor of about 2, usually by a factor of about 3 and more usually by a factor of about 6.

In embodiments where it is desired to modulate, and specifically, reduce the hepatic first-pass metabolism of a drug, the bifunctional molecule generally includes a ligand for an abundant intracellular protein, and more specifically an abundant blood cell intracellular protein, and in certain embodiments an abundant red blood cell or erythrocyte intracellular protein. The binary complex formed between the bifunctional molecule and the intracellular pharmacokinetic modulating protein should be transient, e.g. lasting on average from about 1 to 5, usually 1 to 3 minutes and in many embodiments around two minutes. While a variety of intracellular proteins are of interest, in many embodiments, peptidyl prolyl isomerases, e.g. FKBP and cyclophilin, are preferred as the pharmacokinetic modulating protein. In these embodiments, the amount of drug that is eliminated via hepatic first-pass metabolism is decreased by a factor of at least about 2, including a factor of about 3, usually by at least about 5 and more usually by at least about 10.

In embodiments where it is desired to modulate the volume of distribution of a drug, the bifunctional molecule may include a ligand for a intracellular or extracellular pharmacokinetic modulating protein. Thus, where one wishes to change the distribution of a drug so that the drug is distributed in greater amounts in the intracellular space as compared to the extracellular space, the modulating moiety of the bifunctional molecule is a ligand for an intracellular pharmacokinetic modulating protein, e.g. peptidyl prolyl isomerase, where suitable ligands are described above. Alternatively, where it is desired to enhance the amount of drug located in the extracellular space as compared to that which is present in the intracellular space, the ligand of the bifunctional molecule is a ligand for an extracellular protein, e.g. albumin, where representative ligands and extracellular pharmacokinetic modulating proteins are further described supra.

In certain embodiments, a drug is administered as a bifunctional molecule in order to reduce the degree of blood protein or serum protein, e.g. albumin, binding of the drug. By degree of serum protein binding is meant the propensity of a drug to experience a serum protein effect, i.e. to be bound by serum protein, such as albumin (or another serum protein such as α-1 acid glycoprotein) and be rendered inactive. In this embodiments, the drugs of interest are those drugs which have a certain affinity for the serum protein, e.g. albumin, in their free drug form, where this affinity is generally at least about 10⁻³, usually at least about 10⁻⁵ and more usually at least about 10⁻⁶. In certain embodiments where modulation of serum protein binding effect is desired, the affinity of the modulating ligand for the serum protein will be greater than the affinity of the drug moiety for the serum protein, usually at least about 2 fold greater, in certain embodiments at least about 3 fold greater and in other embodiments at least about 5 fold greater. The linker moiety of the bifunctional molecule is chosen such that the drug moiety of the bifunctional is displayed in manner that retains its desired activity, e.g. target binding activity, when a binary complex is formed with the serum protein, e.g. albumin. In addition, the linker is chosen such that the drug moiety cannot bind to a second serum protein, e.g. albumin molecule, to produce a tripartite complex of two serum protein molecules and a bifunctional molecule. In these embodiments, the degree of albumin binding of the drug or albumin binding effect is reduced by a factor of about 2, usually by a factor of about 3 and more usually by a factor of about 4. As such, the amount of drug that must be administered in order to be effective is generally at least about 2 fold less, in certain embodiments at least about 3 fold less and in other embodiments at least about 4 fold less than the amount of corresponding free drug that must be administered.

The bifunctional molecules identified by the subject screening methods find use in the pharmacological treatment of a host condition, e.g. a disease condition. In the methods of the subject invention, an effective amount of the bifunctional molecule is administered to the host, where “effective amount” means a dosage sufficient to produce the desired result, e.g. an improvement in a disease condition or the symptoms associated therewith. In many embodiments, the amount of drug in the form of the bifunctional molecule that need be administered to the host in order to be an effective amount will vary from that which must be administered in free drug form, where by free drug is meant drug that is not conjugated with another moiety, e.g. as is found in the subject bifunctional molecules. The difference in amounts may vary, and in many embodiments may range from 2 fold to 10 fold. As such, the total drug administered to the subject may be lower, thereby reducing toxicity and side effects experienced by the subject. In certain embodiments, e.g. where the resultant modulated pharmacokinetic property(s) results in enhanced activity as compared to the free drug control, the amount of drug that need be administered to be an effective amount is less than the amount of corresponding free drug that needs to be administered, where the amount may be 2-fold, usually about 4-fold and more usually about 10-fold less than the amount of free drug that is administered.

The bifunctional molecule may be administered to the host using any convenient means capable of producing the desired result. Thus, the bifunctional molecule can be incorporated into a variety of formulations for therapeutic administration. More particularly, the bifunctional molecule of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols. As such, administration of the bifunctional molecule can be achieved in various ways, including oral, buccal, rectal, parenteral, intraperitoneal, intradermal, transdermal, intracheal, etc., administration. In pharmaceutical dosage forms, the bifunctional molecule may be administered alone or in combination with other pharmaceutically active compounds. The following methods and excipients are merely exemplary and are in no way limiting.

For oral preparations, the bifunctional molecules can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The bifunctional molecules can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

The bifunctional molecules can be utilized in aerosol formulation to be administered via inhalation. The compounds of the present invention can be formulated into pressurized acceptable propellants such as dichlorodifluoromethane, propane, nitrogen and the like.

Furthermore, the bifunctional molecules can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition containing active agent. Similarly, unit dosage forms for injection or intravenous administration may comprise the active agent in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

Those of skill will readily appreciate that dose levels can vary as a function of the specific compound, the severity of the symptoms and the susceptibility of the subject to side effects. Preferred dosages for a given compound are readily determinable by those of skill in the art by a variety of means.

The subject methods find use in the treatment of a variety of different disease conditions. In certain embodiments, of particular interest is the use of the subject methods in disease conditions where an active agent or drug having desired activity has been previously identified, but which active agent or drug does not bind to its target with desired affinity and/or specificity. With such active agents or drugs, the subject methods can be used to enhance the binding affinity and/or specificity of the agent for its target.

The specific disease conditions treatable by with the subject bifunctional compounds are as varied as the types of drug moieties that can be present in the bifunctional molecule. Thus, disease conditions include cellular proliferative diseases, such as neoplastic diseases, autoimmune diseases, cardiovascular diseases, hormonal abnormality diseases, infectious diseases, and the like.

By treatment is meant at least an amelioration of the symptoms associated with the disease condition afflicting the host, where amelioration is used in a broad sense to refer to at least a reduction in the magnitude of a parameter, e.g. symptom, associated with the pathological condition being treated, such as inflammation and pain associated therewith. As such, treatment also includes situations where the pathological condition, or at least symptoms associated therewith, are completely inhibited, e.g. prevented from happening, or stopped, e.g. terminated, such that the host no longer suffers from the pathological condition, or at least the symptoms that characterize the pathological condition.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

Kits

Also provided are kits for use in practicing the subject screening methods, as described above. The kits for practicing the subject methods at least include: a metabolizer, such as cytochrome P450 (CYP); and a reporter, such as a fluorogenic CYP substrate. In certain embodiments, the kits may further include a pharmacokinetic modulating protein (PMP) or precusor thereof, e.g., a nucleic acid encoding the PMP. In some embodiments, the PMP is a peptidyl-prolyl isomerase, such as a FKBP or a cyclophilin. In some embodiments, the CYP is CYP3A5, CYP3A4, CYP2E1, CYP2D6, CYP2C19, CYP2C9, CYP2B6, or CYP1A2. The subject kits may further include various buffers, control compounds, standards, and the like for carrying out the subject assays of the present invention.

In certain embodiments, the kits further include at least an information storage and presentation medium that contains control data with which assay results may be compared. The information storage and presentation medium may be in any convenient form, such as printed information on a package insert, an electronic file present on an electronic storage medium, e.g. a magnetic disk, CD-ROM, and the like. In yet other embodiments, the kits may include alternative means for obtaining control data, e.g. a website for obtaining the reference data “on-line.”

The kit components may be present in separate containers, or one or more of the components may be present in the same container, where the containers may be storage containers and/or containers that are employed during the assay for which the kit is designed.

In addition kits with unit doses of the bifunctional molecule, usually in oral or injectable doses and often in a storage stable formulation, are also provided. In such kits, in addition to the containers containing the unit doses will be an informational package insert describing the use and attendant benefits of the drugs in treating pathological condition of interest. Preferred compounds and unit doses are those described herein above.

Systems

Also provided are systems that find use in practicing the subject methods, as described above. For example, in some embodiments, systems for practicing the subject methods may include at least a metabolizer, a reporter and a PMP, as described above. Furthermore, additional reagents that are required or desired in the protocol to be practiced with the system components may be present, as described above.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Synthesis of FKBP-Binding Conjugates

Since curcumin reduces aggregation of amyloid beta and has been shown to have anti-cancer potential, a binfunctional molecule consisting of curcumin and FK506 was generated. Commercial curcumin was coupled to a linker by treatment with C₂H₅ONa, followed by 4-bromoaniline in pyridine. After installation of the primary amine, the curcumin derivative was coupled to the succinimidyl ester of FK506. The activation of FK506 proceeded as previously described (Spencer et al., (1993) Science 262:1019). The product was confirmed by mass spectrometry. The reaction scheme for producing the curcumin-FK506 bifunctional compound is reproduced below:

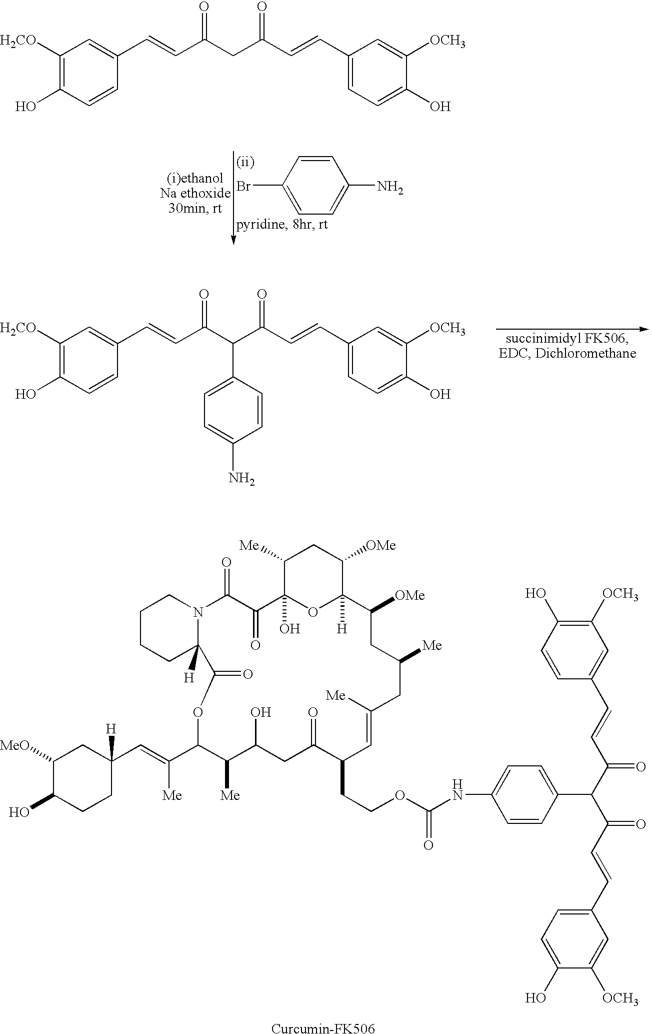

Since TZDM-like molecules have potential as imaging agents and therapeutics for Alzheimer's disease, a bifunctional molecules consisting of TZDM conjugated to a synthetic ligand of FKBP (SLF) was also generated: TZDM was prepared from in refluxing DMSO from the aminothiol and aldehyde (as described above; and as per Zhuang et al (2001) J. Med. Chem. 44:1905). A protected carboxylate was installed by reaction reacting the aryl chloride with the linker in the presence of a Pd catalyst (see Zim and Buchwald (2003) Org. Lett. 5:2413). After deprotection, EDC/NHS were used to create the amide with SLF bearing a pendant primary amine. The product was confirmed by mass spectrometry. The reaction scheme for producing the TZDM-SLF bifunctional compound is reproduced below:

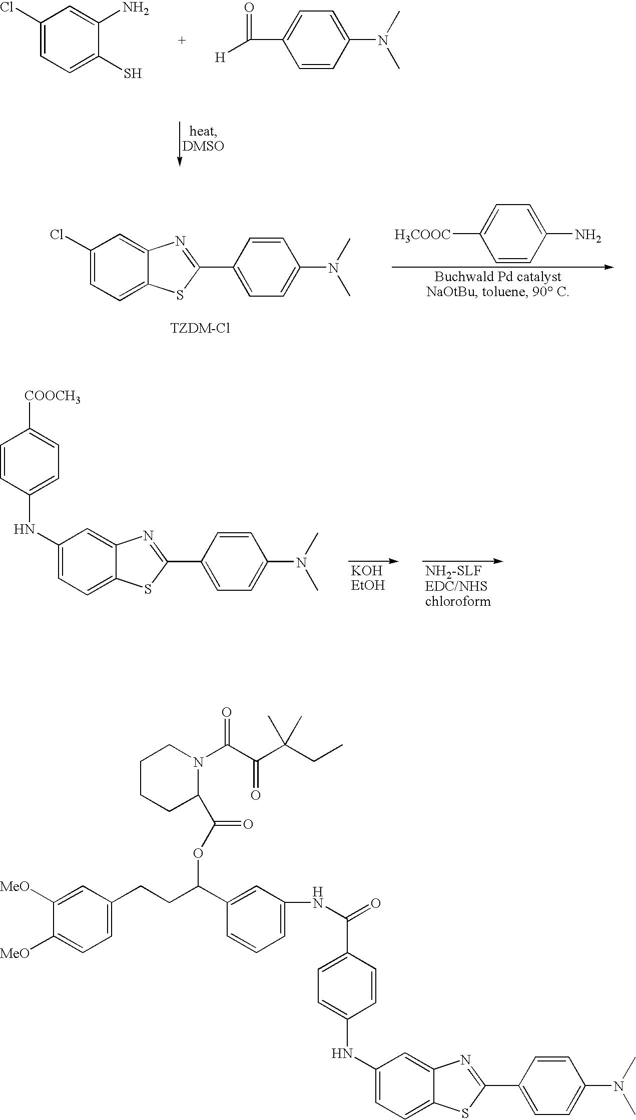

### Examples 2

### Cytochrome P450 Susceptibility Assay

The curcumin-FK506 and TZDM-SLF bifunctional compounds were assayed to determine whether the compounds had at least one modulated pharmacokinetic property. Both the curcumin-FK506 and TZDM-SLF bifunctional compounds include a ligand to the pertidyl-prolyl isomerase FKBP. Therefore, FKBP12 was used in the assay as the PMP. A control was also performed with FK506 in the presence and absence of FKBP12.

The coding sequence of human FKBP12 was subcloned into a pGEX2t vector. Bacteria transformed with this vector produce a fusion protein between FKBP12 and glutathione S-transferase (GST). FKBP12 is purified by standard methods and the GST tag enzymatically removed. Pure FKBP was flash-frozen, and stored at −80° C. at 10-100 micromolar in HEPES or phosphate buffer pH 7.0.

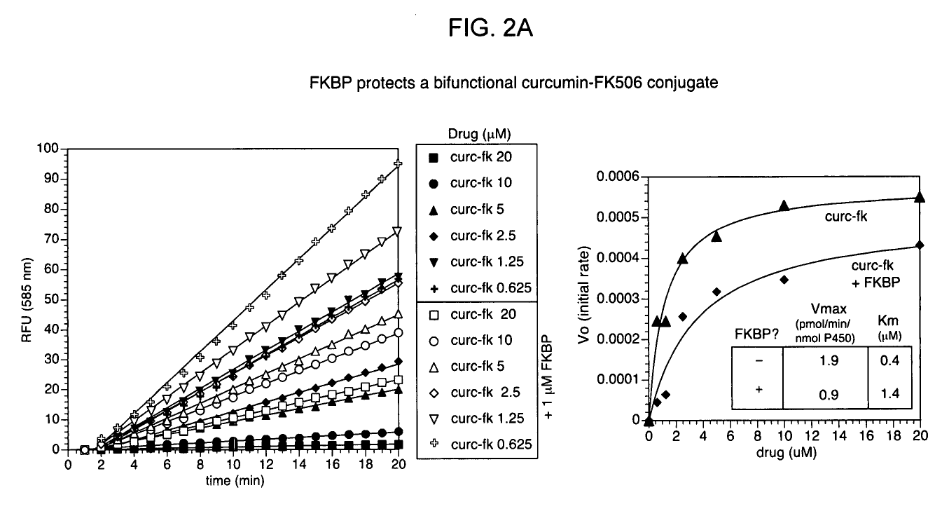

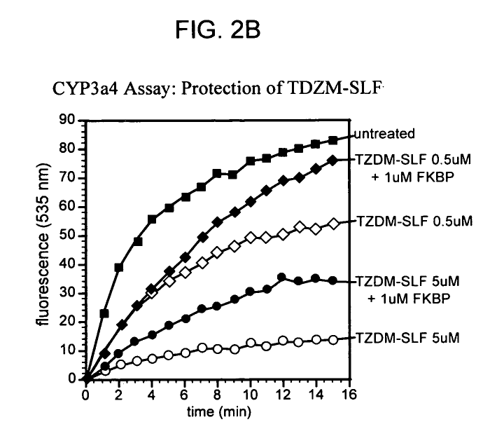

The CYP3A4 compound and the green substrate VIVID® DBOMF (Invitrogen) were used in the assay with the recombinant human FKBP12 (rhFKBP12) and one of the bifunctional compounds. The results of the curcumin-FK506 assay are shown in FIG. 2A, left panel. The results marked as untreated (represented by the closed squares) are control reactions with only the CYP3A4 compound and the green substrate VIVID® DBOMF. Increasing concentrations of the bifunctional compound, curcumin-FK506, were added to this system, either in the presence (open symbols) or absence (closed symbols) of rhFKBP12 at one micromolar. The results show that the fluorescence of the reaction with curcumin-FK506 in the presence of rhFKBP12 was higher as compared to the reaction lacking the rhFKBP12. This is also shown by plotting the initial rate of degradation and calculating the change in Km and Vmax (FIG. 2A, right panel). These results show that this bifunctional molecule is protected from metabolism by binding to the protein partner, FKBP.

The results of the TZDM-SLF assay are shown in FIG. 2B. The results marked as untreated (represented by the closed squares) are control reactions with only the CYP CYP3A4 compound and the green substrate VIVID® DBOMF. The results show that the fluorescence of the reaction with curcumin-FK506 in the presence of rhFKBP 12 (represented by diamonds) was higher as compared to the reaction lacking the rhFKBP12 (represented by open circles). In addition, the results show that the reaction including the bifunctional compound produced a comparable fluorescence to the untreated control showing that the drug moiety of the TZDM-SLF bifunctional molecule is protected and is not metabolized by the CYP.

### Example 3

### Fluorescence Microscopy

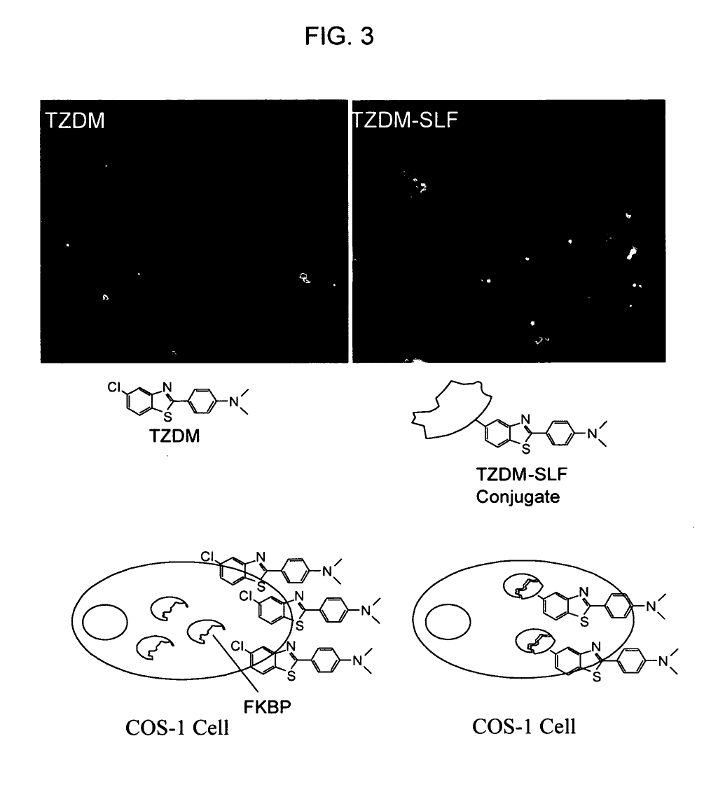

Cellular and tissue distribution of a bifunctional molecule consisting of a FKBP-binding group was also assessed. Such re-distribution is due to changes in the physicochemical characteristics of the new molecule and also is driven by specific, high-affinity interactions with FKBP. For example, the distribution of TZDM and TZDM-SLF were studied in cultured COS-1 cells (see FIG. 3). The intrinsic fluorescence of TZDM was used was a convenient handle for following drug distribution. As shown in FIG. 3 (right panel), TZDM is a lipophilic molecule that resides in membranes when added to COS-1 cells. However, addition of an FKBP-binding group to TZDM reduces membrane insertion in favor of cytoplasmic distribution (see FIG. 3, right panel). This result is consistent with binding to cytoplasmic FKBP12. Matching the distribution of FKBP-binding drug to the known location of a pathogen or target may generate a more effective drug with lower toxicity simply by putting the drug where it is needed. Thus, sequestering FKBP-binding drugs both reduces accessibility to P450 enzymes and also alters the distribution of the bifunctional compounds.

### Example 4

### Cytochrome P450 Susceptibility Assay Using Whole Cells

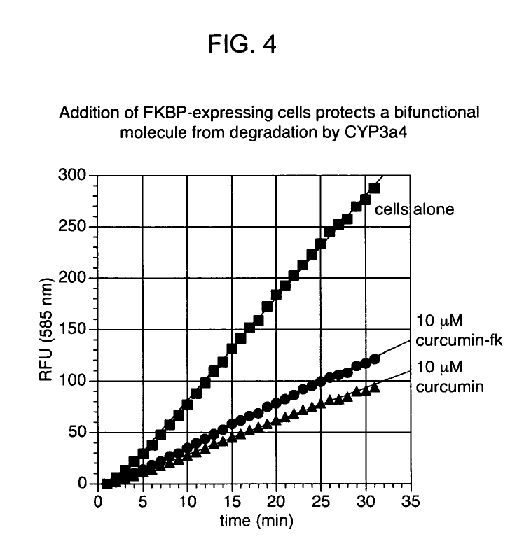

In addition to using rhFKBP12 or other recombinant proteins, whole cells were also tested using the cytochrome P450 assay platform. FIG. 4 shows an experiment in which FKBP-expressing Chinese hamster ovary cells (CHO) were added to the curcumin-FK506 conjugate (circles) described above in the VIVID® assay. Unconjugated curcumin (triangle) was used as a negative control. The graph shows that the bifunctional molecule is protected by addition of the FKBP-expressing cells but that unconjugated curcumin is less protected. The results also show that the absolute fluorescence values typically are higher in these experiments, due to light scatter and intrinsic fluorescence of the cells.

### Example 5

### Protection of FK506 from Metabolism

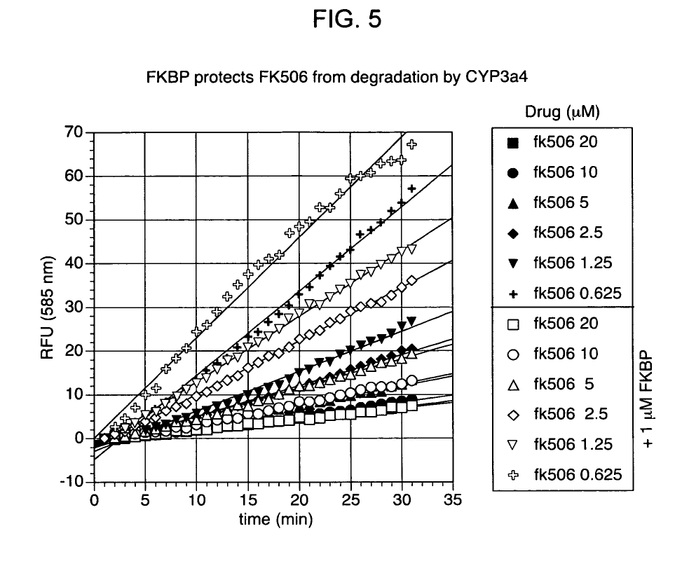

It was next tested whether the protection of FK506 from metabolism could be witnessed in the assay. FK506 is a naturally bifunctional molecule that is known to be protected from degradation in whole animals. However, it has never been tested in an in vitro screen. Therefore, rhFKBP12 and FK506 were added to the VIVID assay. The open symbols are FK506+FKBP and the filled symbols are FK506 alone. As shown in FIG. 5, FK506 was protected from P450 metabolism by the presence of the protein partner. Because FK506 is known to bind FKBP in animals and humans, this data shows that this screening platform can identify compounds that will also be protected in vivo. These results validate the assay platform.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of determining whether a bifunctional molecule comprising a drug moiety has a modulated pharmacokinetic property as compared to a free drug control, comprising:
(a) combining in a reaction mixture:
(i) a metabolizer of said drug,
(ii) a reporter for said metabolizer; and
(iii) said bifunctional molecule, wherein said bifunctional molecule is less than about 5000 daltons and includes said drug or an active derivative thereof and a pharmacokinetic modulating moiety optionally joined by a linking group; and
(b) evaluating said reaction mixture for signal from said reporter substrate to determine whether said bifunctional molecule has a modulated pharmacokinetic property as compared to a free drug control.

2. The method of claim 1, wherein said pharmacokinetic property is half-life, hepatic first-pass metabolism, or volume of distribution.

3. The method of claim 1, wherein said metabolizer of said drug is a cytochrome P450 (CYP).

4. The method of claim 1, wherein said reporter is a fluorescent substrate.

5. The method of claim 4, wherein said CYP is CYP3A5, CYP3A4, CYP2E 1, CYP2D6., CYP2C19. CYP2C9, CYP2B6, or CYP1A2.

6. The method of claim 1, wherein said reaction mixture further includes a pharmacokinetic modulating protein (PMP).

7. The method of claim 1, wherein the PMP is a recombinantly expressed polypeptide or a cell expressing the PMP.

8. The method of claim 6, wherein said PMP is an intracellular protein.

9. The method of claim 6, wherein said PMP is an extracellular protein.

10. The method of claim 6, wherein said PMP is a peptidyl-prolyl isomerase.

11. The method of claim 10, wherein said peptidyl-prolyl isomerase is an FKBP or a cyclophilin.

12. The method of claim 1, wherein said evaluating is over a period of time.

13. The method of claim 1, wherein said method comprises determining whether a plurality of bifunctional molecules for a modulated pharmacokinetic property as compared to free drug controls.

14. A kit for use in screening a bifunctional compound comprising a drug moiety for least one modulated pharmacokinetic property as compared to a free drug control, comprising:
a metabolizer of said drug, a reporter for said metabolizer; and
a pharmacokinetic modulating protein (PMP) or a nucleic acid encoding the PMP.

15. The kit of claim 14, wherein said metabolizer of said drug is a cytochrome P450 (CYP).

16. The kit of claim 14, wherein said reporter is a fluorescent substrate.

17. The kit of claim 15, wherein said CYP is CYP3A5, CYP3A4, CYP2E1, CYP2D6, CYP2C19, CYP2C9, CYP2B6, or CYP1A2.

18. The kit of claim 14, wherein said PMP is an intracellular protein.

19. The kit of claim 14, wherein said PMP is an extracellular protein.

20. The kit of claim 14, wherein said PMP is a peptidyl-prolyl isomerase.

21. The kit of claim 20, wherein said peptidyl-prolyl isomerase is an FKBP or a cyclophilin.

22. A device for use in screening a plurality of bifunctional molecules for a modulated pharmacokinetic property as compared to free drug controls, comprising:
an array of addressable reaction members, wherein each reaction member comprises
a metabolizer of said drug,
a reporter for said metabolizer; and <a pharmacokinetic modulating protein (PMP).

23. The device of claim 22, wherein said metabolizer of said drug is a cytochrome P450 (CYP).

24. The device of claim 22, wherein said reporter is a fluorescent substrate.

25. The device of claim 22, wherein said CYP is CYP3A5, CYP3A4, CYP2E1, CYP2D6, CYP2C19, CYP2C9, CYP2B6, or CYP1A2.

26. The device of claim 22, wherein said PMP is an intracellular protein.

27. The device of claim 22, wherein said PMP is an extracellular protein.

28. The device of claim 22, wherein said PMP is a peptidyl-prolyl isomerase.

29. The device of claim 28, wherein said peptidyl-prolyl isomerase is an FKBP or a cyclophilin.

30. A bifunctional molecule identified by a screening method of claim 1.

31. A bifunctional compound of less than about 5000 daltons comprising of a curcuminoid moiety and a pharmacokinetic modulating moiety, wherein said curcuminoid moiety and said pharmacokinetic modulating moiety are optionally joined by a linking group and said bifunctional molecule exhibits at least one modulated pharmacokinetic property upon administration to a host as compared to a free curcuminoid control.

32. The bifunctional molecule of claim 31, wherein said bifunctional molecule comprises a linking group.

33. The bifunctional compound of claim 31, wherein said curcuminoid moiety is curcumin, demethoxycurcumin, bisdemethoxycurcumin, or tetrahydrocurcumin.

34. The bifunctional molecule of claim 31, wherein said pharmacokinetic modulating moiety binds to a protein.

35. The bifunctional molecule of claim 34, wherein said protein is an extracellular protein.

36. The bifunctional molecule of claim 34, wherein said protein is an intracellular protein.

37. The bifunctional compound of claim 31, wherein said pharmacokinetic modulating moiety is a peptidyl-prolyl isomerase ligand.

38. The bifunctional compound of claim 37, wherein said peptidyl-prolyl isomerase ligand is a ligand for an FKBP or a cyclophilin.

39. The bifunctional compound of claim 37, wherein said peptidyl-prolyl isomerase ligand is FK506, a synthetic ligand of FKBP, or rapamycin.

40. The bifunctional molecule of claim 31, wherein said pharmacokinetic property is selected from the group consisting of half-life, hepatic first-pass metabolism, volume of distribution and degree of blood protein binding.

41. A bifunctional compound of less than about 5000 daltons comprising of a amyloid imaging agent and a pharmacokinetic modulating moiety, wherein said curcuminoid moiety and said pharmacokinetic modulating moiety are optionally joined by a linking group and said bifunctional molecule exhibits at least one modulated pharmacokinetic property upon administration to a host as compared to a free curcuminoid control.

42. The bifunctional molecule of claim 41, wherein said bifunctional molecule comprises a linking group.

43. The bifunctional compound of claim 41, wherein said amyloid imaging agent is TZDM.

44. The bifunctional molecule of claim 41, wherein said pharmacokinetic modulating moiety binds to a protein.

45. The bifunctional molecule of claim 44, wherein said protein is an extracellular protein.

46. The bifunctional molecule of claim 44, wherein said protein is an intracellular protein.

47. The bifunctional compound of claim 41, wherein said pharmacokinetic modulating moiety is a peptidyl-prolyl isomerase ligand.

48. The bifunctional compound of claim 47, wherein said peptidyl-prolyl isomerase ligand is a ligand for an FKBP or a cyclophilin.

49. The bifunctional molecule of claim 47, wherein said peptidyl-prolyl isomerase ligand is FK506, a synthetic ligand of FKBP, or rapamycin.

50. The bifunctional compound of claim 41, wherein said pharmacokinetic property is selected from the group consisting of half-life, hepatic first-pass metabolism, volume of distribution and degree of blood protein binding.
